(19) United States
(12) Patent Application Publication
(10) Pub. No.: US 2025/0082584 A1
(43) Pub. Date: Mar. 13, 2025

(54) TRANSDERMAL THERAPEUTIC SYSTEM FOR THE TRANSDERMAL ADMINISTRATION OF SELEXIPAG

(71) Applicant: LTS LOHMANN THERAPIE-SYSTEME AG, Andernach (DE)

(72) Inventors: Florian HAMMES, Andernach (DE); Anja TOMELERI, Neuwied (DE); Tobias KLEUDGEN, Ettringen (DE)

(21) Appl. No.: 18/728,006

(22) PCT Filed: Jan. 26, 2023
(86) PCT No.: PCT/EP2023/051945

§ 371 Date: Jul. 10, 2024

(30) Foreign Application Priority Data
Jan. 27, 2022 (EP) 22153580.0

(51) Int. Cl. A61K 9/70; A61K 31/4965

(52) U.S. Cl. CPC A61K 9/7053; A61K 31/4965

(57) ABSTRACT

The present invention relates to transdermal therapeutic systems (TTS) for the transdermal administration of selexipag.

### TECHNICAL FIELD OF THE INVENTION

The present invention relates to a transdermal therapeutic system (TTS) for the transdermal administration of selexipag, processes of manufacture, and uses thereof.

### BACKGROUND OF THE INVENTION

The active ingredient selexipag (also known as e.g. 2-[4-[(5,6-diphenylpyrazin-2-yl)-propan-2-ylamino]butoxy]-N-methylsulfonylacetamide or CAS No. 475086-01-2), is a medicament used for e.g. the treatment of pulmonary arterial hypertension (PAH). Selexipag and its active metabolite ACT-333679 (also known as MRE-269) binds to the prostacyclin receptor, which leads to vasodilation in the pulmonary circulation. Selexipag (left) and its active metabolite ACT-333679 (the free carboxylic acid) in the form of its free base (right) have the following chemical formulae.

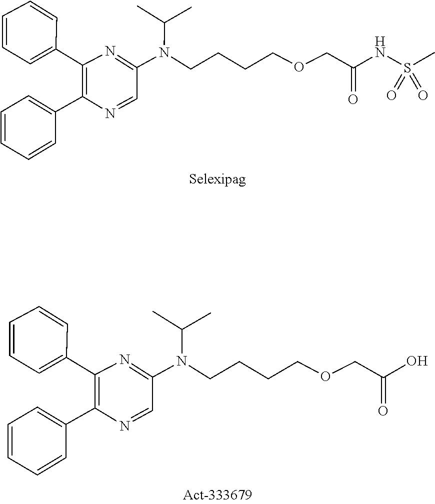 [SMILES: CC(C)N(CCCCOCC(=O)NS(C)(=O)=O)c1cnc(-c2ccccc2)c(-c2ccccc2)n1 ; CC(C)N(CCCCOCC(=O)O)c1cnc(-c2ccccc2)c(-c2ccccc2)n1]

Selexipag can be used for the treatment of pulmonary arterial hypertension (PAH).

So far, selexipag can be administered via the oral route or via the intravenous route. However, there is currently no commercial TTS for the transdermal administration of selexipag.

One challenge in developing a TTS for the transdermal administration of selexipag is the permeation through the skin of selexipag. This challenge was addressed in US 2019/0099383 A1 by developing a patch comprising ACT-333679 in the form of its free base. Skin permeability was achieved with a composition comprising ACT-333679. However, this composition does not comprise selexipag. In addition, the provision of an ACT-333679-containing patch is not exemplified.

Against this background, there is a need in the art for a TTS for the transdermal administration of selexipag.

### OBJECTS AND SUMMARY OF THE INVENTION

It is an object of certain embodiments of the present invention to provide a TTS for the transdermal administration of selexipag.

It is a further object of certain embodiments of the present invention to provide a TTS for the transdermal delivery of selexipag, wherein said TTS has a simple structure, e.g. including less than 15, less than 10, or less than 6 components, for said transdermal delivery.

It is a further object of certain embodiments of the present invention to provide a TTS for the transdermal delivery of selexipag, wherein said TTS can be produced in a constant quality.

It is a further object of certain embodiments of the present invention to provide a TTS for the transdermal administration of selexipag, which provides a permeation rate, which is sufficient for achieving a therapeutically effective dose over a prolonged administration period such as over an administration period, such as over 48 hours, or over 72 hours, or over 84 hours, or over 168 hours, preferably over 168 hours.

It is a further object of certain embodiments of the present invention to provide a TTS for the transdermal administration of selexipag, which provides an improved permeation rate over a prolonged administration period such as over an administration period, such over 48 hours, or over 72 hours, or over 84 hours or over 168 hours, preferably over 168 hours.

It is a further object of the present invention to provide a TTS for the transdermal administration of selexipag, with a high active-agent utilization, i.e. a TTS, which does not require a high excess amount of active agent to provide a sufficient release performance during an administration period.

It is a further object of certain embodiments of the present invention to provide a TTS for the transdermal administration of selexipag, which is suitable for use in a method of treating a human patient.

It is an object of certain embodiments of the present invention to provide a TTS for the transdermal administration of selexipag, wherein said TTS is easy to manufacture.

At least one of these objects and others are accomplished by the present invention which, according to a first aspect, is directed to a transdermal therapeutic system for the transdermal administration of selexipag comprising a self-adhesive selexipag-containing layer structure, said self-adhesive selexipag-containing layer structure comprising:
A) a backing layer;
B) a selexipag-containing layer comprising:
   1. selexipag;
   2. at least one polar; and
   3. at least one nonpolar polymer.

According to a second aspect, the present invention is directed to a transdermal therapeutic system according to the first aspect for use in a method of treating a human patient.

### Definitions

Within the meaning of this invention, the term “transdermal therapeutic system” (TTS) refers to a system by which the active agent (e.g. selexipag) is administered to the systemic circulation via transdermal delivery and refers to the entire individual dosing unit that is applied, after removing an optionally present release liner, to the skin of a patient, and which comprises a therapeutically effective amount of active agent in an active agent-containing layer structure and optionally an additional adhesive overlay on top of the active agent-containing layer structure. The active agent-containing layer structure may be located on a release liner (a detachable protective layer), thus, the TTS may further comprise a release liner. Within the meaning of this invention, the term “TTS” in particular refers to systems providing transdermal delivery, excluding active delivery for example via iontophoresis or microporation. Transdermal therapeutic systems may also be referred to as transdermal drug delivery systems (TDDS) or transdermal delivery systems (TDS).

Within the meaning of this invention, the term “selexipag-containing layer structure” refers to the layer structure containing a therapeutically effective amount of selexipag and comprises a backing layer and at least one active agent-containing layer. Preferably, the selexipag-containing layer structure is a selexipag-containing self-adhesive layer structure.

Within the meaning of this invention, the term “therapeutically effective amount” refers to a quantity of active agent in the TTS which is, if administered by the TTS to a patient, sufficient to provide e.g. a symptomatic treatment of pulmonary arterial hypertension (PAH) or chronic thromboembolic pulmonary hypertension (CTEPH). A TTS usually contains more active in the system than is in fact provided to the skin and the systemic circulation. This excess amount of active agent is usually necessary to provide enough driving force for the delivery from the TTS to the systemic circulation. In it to be understood that the selexipag-containing layer according to the present invention comprises selexipag in a therapeutically effective amount.

Within the meaning of this invention, the terms “active”, “active agent”, and the like, as well as the term “selexipag” refer to selexipag in any pharmaceutically acceptable chemical and morphological form and physical state. These forms include without limitation cocrystals, solvates, hydrates, clathrates, and so on, as well as selexipag in the form of particles which may be micronized, crystalline and/or amorphous, and any mixtures of the aforementioned forms. The selexipag, where contained in a medium such as a solvent, may be dissolved or dispersed or in part dissolved and in part dispersed.

Within the meaning of this invention, the term “selexipag” refers to selexipag in any pharmaceutically acceptable chemical and morphological form and physical state.

Unless otherwise indicated, in particular the amount of selexipag in the layer structure relates to the amount of selexipag included in the TTS during manufacture of the TTS and is calculated based on selexipag. E.g., when 0.1 mmol (equal to 49.7 mg) selexipag is included in the TTS during manufacture, the amount of selexipag in the layer structure is, within the meaning of the invention, 0.1 mmol or 49.7 mg.

The selexipag starting material included in the TTS during manufacture of the TTS may be in the form of particles. Selexipag may e.g. be present in the active agent-containing layer structure in the form of particles and/or be dissolved.

Within the meaning of this invention, the term “particles” refers to a solid, particulate material comprising individual particles, the dimensions of which are negligible compared to the material. In particular, the particles are solid, including plastic/deformable solids, including amorphous and crystalline materials.

Within the meaning of this invention, the term “dispersing” refers to a step or a combination of steps wherein a starting material (e.g. selexipag) is not totally dissolved. Dispersing in the sense of the invention comprises the dissolution of a part of the starting material (e.g. selexipag particles), depending on the solubility of the starting material (e.g. the solubility of selexipag in the coating composition).

There are two main types of TTS for active agent delivery, i.e. matrix-type TTS and reservoir-type TTS. The release of the active agent in a matrix-type TTS is mainly controlled by the matrix including the active agent itself. In contrast, thereto, a reservoir-type TTS typically needs a rate controlling membrane controlling the release of the active agent. In principle, also a matrix-type TTS may contain a rate-controlling membrane. However, matrix-type TTS are advantageous in that, compared to reservoir-type TTS, usually no rate determining membranes are necessary and no dose dumping can occur due to membrane rupture. In summary, matrix-type transdermal therapeutic systems (TTS) are less complex in manufacture, easy, and convenient to use by patients.

Within the meaning of this invention, “matrix-type TTS” refers to a system or structure wherein the active is homogeneously dissolved and/or dispersed within a polymeric carrier, i.e. the matrix, which forms with the active agent and optionally remaining ingredients a matrix layer. In such a system, the matrix layer controls the release of the active agent from the TTS. Preferably, the matrix layer has sufficient cohesion to be self-supporting so that no sealing between other layers is required. Accordingly, the active agent-containing layer may in one embodiment of the invention be an active agent-containing matrix layer, wherein the active agent is homogeneously distributed within a polymer matrix. In certain embodiments, the active agent-containing matrix layer may comprise two active agent-containing matrix layers, which may be laminated together. Matrix-type TTS may in particular be in the form of a “drug-in-adhesive”-type TTS referring to a system wherein the active is homogeneously dissolved and/or dispersed within a pressure-sensitive adhesive matrix. In this connection, the active agent-containing matrix layer may also be referred to as active agent-containing pressure sensitive adhesive layer or active agent-containing pressure sensitive adhesive matrix layer. A TTS comprising the active agent dissolved and/or dispersed within a polymeric gel, e.g. a hydrogel, is also considered to be of matrix-type in accordance with present invention.

TTS with a liquid active agent-containing reservoir are referred to by the term “reservoir-type TTS”. In such a system, the release of the active agent is preferably controlled by a rate-controlling membrane. In particular, the reservoir is sealed between the backing layer and the rate-controlling membrane. Accordingly, the active agent-containing layer may in one embodiment be an active agent-containing reservoir layer, which preferably comprises a liquid or semi-liquid reservoir comprising the active agent. Furthermore, the reservoir-type TTS typically additionally comprises a skin contact layer, wherein the reservoir layer and the skin contact layer may be separated by the rate-controlling membrane. In the reservoir layer, the active agent is preferably dissolved in a solvent such as ethanol or water or in silicone oil. The skin contact layer typically has adhesive properties.

Reservoir-type TTS are not to be understood as being of matrix-type within the meaning of the invention. However, microreservoir TTS (biphasic systems having deposits (e.g. spheres, droplets) of an inner active-containing phase dispersed in an outer polymer phase), considered in the art to be a mixed from of a matrix-type TTS and a reservoir-type TTS that differ from a homogeneous single phase matrix-type TTS and a reservoir-type TTS in the concept of drug transport and drug delivery, are considered to be of matrix-type within the meaning of the invention. The sizes of microreservoir droplets can be determined by an optical microscopic measurement (for example by Leica MZ16 including a camera, for example Leica DSC320) by taking pictures of the microreservoirs at different positions at an enhancement factor between 10 and 400 times, depending on the required limit of detection. By using imaging analysis software, the sizes of the microreservoirs can be determined.

Within the meaning of this invention, the term “selexipag-containing layer” refers to a layer containing selexipag and providing the area of release. The term covers selexipag-containing matrix layers and selexipag-containing reservoir layers. If the selexipag-containing layer is a selexipag-containing matrix layer, said layer is present in a matrix-type TTS. If the polymer is a pressure-sensitive adhesive, the matrix layer may also represent the adhesive layer of the TTS, so that no additional skin contact layer is present. Alternatively, an additional skin contact layer may be present as adhesive layer, and/or an adhesive overlay is provided. The additional skin contact layer is typically manufactured such that it is selexipag-free. However, due to the concentration gradient, selexipag will migrate from the matrix layer to the additional skin contact layer over time, until equilibrium is reached. The additional skin contact layer may be present on the selexipag-containing matrix layer or separated from the selexipag-containing matrix layer by a membrane, preferably a rate controlling membrane. Preferably, the selexipag-containing matrix layer has sufficient adhesive properties, so that no additional skin contact layer is present. If the selexipag-containing layer is a selexipag-containing reservoir layer, said layer is present in a reservoir-type TTS, and the layer comprises selexipag in a liquid reservoir. In addition, an additional skin contact layer is preferably present, in order to provide adhesive properties. Preferably, a rate-controlling membrane separates the reservoir layer from the additional skin contact layer. The additional skin contact layer can be manufactured such that it is selexipag-free or selexipag-containing. If the additional skin contact layer is free of active agent the active agent will migrate, due to the concentration gradient, from the reservoir layer to the skin contact layer over time, until equilibrium is reached. Additionally an adhesive overlay may be provided.

As used herein, the selexipag-containing layer is preferably a selexipag-containing matrix layer, and it is referred to the final solidified layer. Preferably, a selexipag-containing matrix layer is obtained after coating and drying the solvent-containing coating composition as described herein. Alternatively a selexipag-containing matrix layer is obtained after melt-coating and cooling. The selexipag-containing matrix layer may also be manufactured by laminating two or more such solidified layers (e.g. dried or cooled layers) of the same composition to provide the desired area weight. The matrix layer may be self-adhesive (in the form of a pressure sensitive adhesive matrix layer), or the TTS may comprise an additional skin contact layer of a pressure sensitive adhesive for providing sufficient tack. Preferably, the matrix layer is a pressure sensitive adhesive matrix layer. Optionally, an adhesive overlay may be present.

Within the meaning of this invention, the term “pressure-sensitive adhesive” (also abbreviated as “PSA”) refers to a material that in particular adheres with finger pressure, is permanently tacky, exerts a strong holding force and should be removable from smooth surfaces without leaving a residue. A pressure sensitive adhesive layer, when in contact with the skin, is “self-adhesive”, i.e. provides adhesion to the skin so that typically no further aid for fixation on the skin is needed. A “self-adhesive” layer structure includes a pressure sensitive adhesive layer for skin contact, which may be provided in the form of a pressure sensitive adhesive matrix layer or in the form of an additional layer, i.e. a pressure sensitive adhesive skin contact layer. An adhesive overlay may still be employed to advance adhesion. The pressure-sensitive adhesive properties of a pressure-sensitive adhesive depend on the polymer or polymer composition (e.g. a mixture of a silicone and an acrylic polymer; or a combination of a polyisobutylene mixture and a polyvinylpyrrolidone) used. In particular, the pressure-sensitive adhesive according to the present invention may comprise at least one polyvinylpyrrolidone and at least one nonpolar polymer.

Within the meaning of this invention, the term “polymer” refers to any substance consisting of so-called repeating units obtained by polymerizing one or more monomers, and includes homopolymers which consist of one type of monomer and copolymers which consist of two or more types of monomers. Polymers may be of any architecture such as linear polymers, star polymer, comb polymers, brush polymers, of any monomer arrangements in case of copolymers, e.g. alternating, statistical, block copolymers, or graft polymers. The minimum molecular weight varies depending on the polymer type and is known to the skilled person. Polymers may e.g. have a molecular weight above 2000, preferably above 5000 and more preferably above 10,000 Dalton. Correspondingly, compounds with a molecular weight below 2000, preferably below 5000 or more preferably below 10,000 Dalton are usually referred to as oligomers.

Within the meaning of this invention, the term “polar polymer” is used for those polymers which preferably dissolve in, or are swollen by, water. Polar polymers are preferably essentially composed of monomer units including polar functional groups which are polar such as hydroxy-, carbonyl-, carboxyl-, amino-, quaternary ammonium-, sulfhydryl-, phosphate-, sulfate groups and certain carboxylic esters. The term polar polymers encompasses polar acrylic polymers and polyvinylpyrrolidones.

Within the meaning of this invention, the term “nonpolar polymer” is used for those polymers which neither dissolve in, nor are swollen by, water. Nonpolar polymers are essentially composed of monomers which are nonpolar. Nonpolar polymers encompass materials such as polyethylene, polyisobutylene, polystyrene, polyvinylchloride, polytetrafluorethylene, or polysiloxanes.

Within the meaning of this invention, the term “skin contact layer” refers to the layer included in the active agent-containing layer structure to be in direct contact with the skin of the patient during administration. This may be the active agent-containing layer. When the TTS comprises an additional skin contact layer, the other layers of the active agent-containing layer structure do not contact the skin and do not necessarily have self-adhesive properties. As outlined above, an additional skin contact layer attached to the active agent-containing layer may over time absorb parts of the active agent. An additional skin contact layer may be used to enhance adherence. The sizes of an additional skin contact layer and the active agent-containing layer are usually coextensive and correspond to the area of release. However, the area of the additional skin contact layer may also be greater than the area of the active agent-containing layer. In such a case, the area of release still refers to the area of the active agent-containing layer.

Within the meaning of this invention, the term “area weight” refers to the dry weight of a specific layer, e.g. of the matrix layer, provided in g/m². The area weight values are subject to a tolerance of ±10%, preferably ±7.5%, due to manufacturing variability.

If not indicated otherwise “%” refers to % by weight.

Within the meaning of this invention, the term “cross-linking agent” refers to a substance which is able to cross-link functional groups contained within the polymer.

Within the meaning of this invention, the term “adhesive overlay” refers to a self-adhesive layer structure that is free of active agent and larger in area than the active agent-containing structure and provides additional area adhering to the skin, but no area of release of the active agent. It enhances thereby the overall adhesive properties of the TTS. The adhesive overlay comprises a backing layer that may provide occlusive or non-occlusive properties and an adhesive layer. Preferably, the backing layer of the adhesive overlay provides non-occlusive properties.

Within the meaning of this invention, the term “backing layer” refers to a layer which supports the active agent-containing layer or forms the backing of the adhesive overlay. At least one backing layer in the TTS and usually the backing layer of the active agent-containing layer is substantially impermeable to the active agent contained in the layer during the period of storage and administration and thus prevents active loss or cross-contamination in accordance with regulatory requirements. Preferably, the backing layer is also occlusive, meaning substantially impermeable to water and water-vapor. Suitable materials for a backing layer include polyethylene terephthalate (PET), polyethylene (PE), ethylene vinyl acetate-copolymer (EVA), polyurethanes, and mixtures thereof. Suitable backing layers are thus for example PET laminates, EVA-PET laminates and PE-PET laminates. Also suitable are woven or non-woven backing materials.

The TTS according to the present invention can be characterized by certain parameters as measured in an in vitro skin permeation test.

Where not otherwise indicated, the in vitro permeation test is performed in a Franz diffusion cell with dermatomed split-thickness human skin with a thickness of 500 μm and an intact epidermis, and with an aqueous buffer comprising 0.9% aqueous sodium chloride, 3% vanillin, 0.5% methyl-β-cyclodextrin, 30% acetonitrile as receptor medium (32° C.).

The amount of active permeated into the receptor medium is determined in regular intervals using a validated HPLC method with a UV photometric detector by taking a sample volume. The receptor medium is completely or in part replaced by fresh medium when taking the sample volume, and the measured amount of active permeated relates to the amount permeated between the two last sampling points and not the total amount permeated so far.

Thus, within the meaning of this invention, the parameter “permeated amount” is provided in μg/cm² and relates to the amount of active permeated in a sample interval at certain elapsed time. E.g., in an in vitro permeation test as described above, wherein the amount of active permeated into the receptor medium has been e.g. measured at hours 0, 8, 24, 36, 48, 72, 104, 144, and 168, the “permeated amount” of active can be given e.g. for the sample interval from hour 36 to hour 48 and corresponds to the measurement at hour 48, wherein the receptor medium has been exchanged completely at hour 36.

The permeated amount can also be given as a “cumulative permeated amount”, corresponding to the cumulated amount of active permeated at a certain point in time. E.g., in an in vitro permeation test as described above, wherein the amount of active permeated into the receptor medium has been e.g. measured at hours 0, 3, 6, 8, 24 and 32, the “cumulative permeated amount” of active at hour 32 corresponds to the sum of the permeated amounts from hour 0 to hour 3, hour 3 to hour 6, hour 6 to hour 8, hour 8 to 24 and hour 24 to hour 32.

Within the meaning of this invention, the parameter “skin permeation rate” for a certain sample interval at certain elapsed time is provided in μg/cm²-hr and is calculated from the permeated amount in said sample interval as measured by in vitro permeation test as described above in μg/cm², divided by the hours of said sample interval. E.g. the skin permeation rate in an in vitro permeation test as described above, wherein the amount of active permeated into the receptor medium has been e.g. measured at hours 0, 8, 24, 36, 48, 72, 104, 144, and 168, the “skin permeation rate” at hour 48 is calculated as the permeated amount in the sample interval from hour 36 to hour 48 divided by 12 hours.

A “cumulative skin permeation rate” can be calculated from the respective cumulative permeated amount by dividing the cumulative permeated amount by the elapsed time. E.g. in an in vitro permeation test as described above, wherein the amount of active permeated into the receptor medium has been e.g. measured at hours 0, 8, 24, 32, 48, 72, 104, 144, and 168, the “cumulative skin permeation rate” at hour 48 is calculated as the cumulative permeated amount at hour 48 (see above) divided by 48 hours, unless indicated otherwise.

Within the meaning of this invention, the above parameter “permeated amount” and “skin permeation rate” (as well as “cumulative permeated amount” and “cumulative skin permeation rate”) refer to mean values calculated from at least 3 in vitro permeation test experiments. Where not otherwise indicated, the standard deviation (SD) of these mean values refer to a corrected sample standard deviation, calculated using the formula:

$$SD = \sqrt{\frac{1}{n - 1} {\sum }_{i = 1}^{n} {( {x}_{i} - \overline{x} )}^{2}}$$

wherein n is the sample size, {x₁, x₂, . . . xₙ} are the observed values and x is the mean value of the observed values.

Within the meaning of this invention, the term “room temperature” refers to the unmodified temperature found indoors in the laboratory where the experiments are conducted and usually lies within 15 to 35° C., preferably about 18 to 25° C.

Within the meaning of this invention, the term “dissolve” refers to the process of obtaining a solution, which is clear and does not contain any particles, as visible to the naked eye.

Within the meaning of this invention, the term “extended period of time” relates to a period of at least or about 24 h, at least or about 48 h, at least or about 84 h, at least or about 168 h, at least or about 1 day, at least or about 2 days, at least or about 3.5 days, or at least or about 7 days, or to a period of about 24 h to about 168 h or about 1 to about 7 day(s), or about 48 h to about 168 h or about 2 to about 7 days.

Within the meaning of this invention, the term “solvent” refers to any liquid substance, which preferably is a volatile organic liquid such as methanol, ethanol, isopropanol, acetone, ethyl acetate, methylene chloride, hexane, n-heptane, toluene and mixtures thereof.

Within the meaning of this invention, the term “additive” refers to any component of a selexipag-containing layer within a TTS according to the present invention besides selexipag, at least one polar polymer, and at least one nonpolar polymer.

Within the meaning of this invention, the term “patient” refers to a subject who has presented a clinical manifestation of a particular symptom or symptoms suggesting the need for treatment, who is treated preventatively or prophylactically for a condition, or who has been diagnosed with a condition to be treated.

### BRIEF DESCRIPTION OF THE DRAWINGS

FIG. 1 depicts the skin permeation rate of selexipag (also known as A Flux) for TTS prepared according to Example 1 (Formulations 1 to 3) over a time interval of 168 hours.

FIG. 2 depicts the skin permeation rate of selexipag for TTS prepared according to Example 2 (Formulations 4 to 7) over a time interval of 168 hours.

FIG. 3 depicts the skin permeation rate of selexipag for TTS prepared according to Example 3 (Formulations 8 to 12) over a time interval of 168 hours.

FIG. 4 depicts the skin permeation rate of selexipag for TTS prepared according to Example 4 (Formulations 13 to 15) over a time interval of 168 hours.

FIG. 5 depicts the skin permeation rate of selexipag for TTS prepared according to Example 5 (Formulations 16 to 19) over a time interval of 168 hours.

FIG. 6 depicts the skin permeation rate of selexipag for TTS prepared according to Example 6 (Formulations 20 to 25) over a time interval of 168 hours.

FIG. 7 depicts the cumulative permeated amount of MRE-269 (also known as ACT-333679) for Compositions prepared according to Example 9 (Compositions 1 and 2) over a time interval of 168 hours.

FIG. 8 depicts the cumulative permeated amount of selexipag for Compositions prepared according to Example 9 (Compositions 1 and 2) over a time interval of 168 hours.

### DETAILED DESCRIPTION

### TTS Structure

The present invention relates to a transdermal therapeutic system for the transdermal administration of selexipag comprising a selexipag-containing layer structure.

The selexipag-containing layer structure according to the invention comprises A) a backing layer and B) a selexipag-containing layer comprising a therapeutically effective amount of selexipag at least one polar polymer, and at least one nonpolar polymer. The selexipag-containing layer structure is preferably a selexipag-containing self-adhesive layer structure.

The backing layer is preferably substantially selexipag-impermeable. Furthermore, it is preferred that the backing layer is occlusive as outlined above.

The selexipag-containing layer may be directly attached to the backing layer, so that no further layer between the backing layer and the selexipag-containing layer is present.

The TTS according to the present invention may be a matrix-type TTS or a reservoir-type TTS, and preferably is a matrix-type TTS.

The selexipag-containing layer structure according to the invention is normally located on a detachable protective layer (release liner), from which it is removed immediately before application to the surface of the patient's skin. Thus, the TTS may further comprise a release liner. A TTS protected this way is usually stored in a blister pack or a seam-sealed pouch. The packaging may be child resistant and/or senior friendly.

In a preferred embodiment of the present invention, the selexipag-containing layer is a selexipag-containing pressure sensitive adhesive layer and represents the skin contact layer. That is, the selexipag-containing layer structure does not comprise an additional skin contact layer attached to the selexipag-containing layer. In this connection, the selexipag-containing layer is preferably a selexipag-containing matrix layer, which is self-adhesive. The self-adhesive properties of the selexipag-containing layer structure are preferably provided by the at least one polar polymer and/or the at least nonpolar polymer. Thus, in a preferred embodiment of the invention, the at least one polar polymer and/or the at least one nonpolar polymer is a pressure sensitive adhesive. Further details regarding the selexipag-containing layer and the at least one polar and nonpolar polymer according to the invention are provided further below.

In another embodiment of the present invention, the selexipag-containing layer structure further comprises an additional skin contact layer. The skin contact layer is preferably self-adhesive and provides adhesive properties. Thus, in one embodiment of the present invention, the selexipag-containing layer structure further comprises C) a skin contact layer on the selexipag-containing layer. In this connection, the additional skin contact layer may also contain at least one polar polymer and/or at least one nonpolar polymer. For example, when the additional skin contact layer comprises a pressure-sensitive adhesive based on polysiloxanes and acrylic polymers, the selexipag-containing layer may comprise the same pressure-sensitive adhesive based on polysiloxanes and acrylic polymers, or a different pressure-sensitive adhesive based on polysiloxanes and acrylic polymers or a different polymer. The additional skin contact layer is preferably obtainable by coating and drying an adhesive coating composition.

In certain embodiments of the invention, wherein the selexipag-containing layer structure comprises an additional skin contact layer, the additional skin contact layer has an area weight of from about 10 to about 160 g/m², from about 10 to about 100 g/m², or from about 10 to about 60 g/m². The total amount of polymer contained in the skin contact layer may range from about 40% to about 100% by weight, preferably from about 50% to about 100% by weight, more preferably from about 60% to about 100% by weight based on the skin contact layer. The skin contact layer may comprise an active agent. The active agent may be selexipag, as well. The active agent in the skin contact layer may also be an additional active agent reasonable for an administration together with selexipag. In a preferred embodiment, the skin contact layer is free of active agent, that is, is prepared without the addition of an active agent.

According to certain embodiments of the invention, the TTS may further comprise an adhesive overlay. This adhesive overlay is in particular larger in area than the selexipag-containing structure and is attached thereto for enhancing the adhesive properties of the overall transdermal therapeutic system. Said adhesive overlay comprises a backing layer and an adhesive layer. The adhesive overlay provides additional area adhering to the skin but does not add to the area of release of the selexipag. The adhesive overlay comprises a self-adhesive polymer or a self-adhesive polymer mixture selected from the group consisting of silicone acrylic hybrid polymers, acrylic polymers, polysiloxanes, polyisobutylenes, and mixtures thereof, which may be identical to or different from any polymer or polymer mixture included in the selexipag-containing layer structure. In one embodiment, the TTS is free of an adhesive overlay on top of the selexipag-containing layer structure.

Depending on the dosage, the area of release of the TTS ranges from about 1 cm² to about 150 cm², preferably from about 5 cm² to about 130 cm², more preferably from about 10 cm² to less than 120 cm².

The TTS according to the invention may further comprise one or more anti-oxidants. The anti-oxidants may be contained in the selexipag-containing layer or in an additional skin contact layer or in both the selexipag-containing layer and the additional skin contact layer. Suitable anti-oxidants are sodium metabisulfite, ascorbyl palmitate, tocopherol and esters thereof, ascorbic acid, butylhydroxytoluene, butylhydroxyanisole or propyl gallate, preferably butylhydroxytoluene, ascorbyl palmitate and tocopherol. The anti-oxidants may be conveniently present in the selexipag-containing layer, preferably in an amount of from about 0.001 to about 1.0% of the selexipag-containing layer, more preferably in an amount of from about 0.02 to about 0.5% of the selexipag-containing layer.

The TTS according to the invention may further comprise in addition to the above mentioned ingredients at least one excipient or further component, for example from the group of cross-linking agents, solubilizers, fillers, tackifiers, film-forming agents, plasticizers, stabilizers, softeners, substances for skincare, permeation enhancers, pH regulators, and preservatives. In one preferred embodiment of the present invention, no additional excipients or additives are required.

### Selexipag-Containing Layer

As outlined in more detail above, the TTS according to the present invention comprises a selexipag-containing layer structure comprising a selexipag-containing layer. The selexipag-containing layer according to the invention comprises a therapeutically effective amount of selexipag; at least one polar polymer; and at least one nonpolar polymer. Such composition of a selexipag-containing layer contributes to a TTS having a low complexity in structure, as the selexipag-containing layer only includes a little amount of components and, at the same time, provides sufficient release characteristics. Further details on the sufficient “release characteristics” can be found in the respective section and in the Examples section below.

According to certain embodiments, a content of selexipag in the selexipag-containing layer ranges from about 0.1 to about 10%, preferably from about 0.1 to about 8.0%, more preferably from about 0.3 to about 6.5%, and in particular from about 0.5 to about 5.5%, by weight based on the total weight of the selexipag-containing layer. It is preferred that said content of selexipag ranges from about 0.5 to about 3.0% by weight based on the total weight of the selexipag-containing layer. Alternatively, it is preferred that said content of selexipag ranges from about more than 3.0 to about 6% by weight based on the total weight of the selexipag-containing layer.

According to certain embodiments, the selexipag-containing layer further comprises an enhancer, wherein said enhancer is preferably selected from the group consisting of tetrahydrofurfuryl alcohol polyethylene glycol ether, levulinic acid, transcutol, lauryl lactate, methyl laurate, dihydrolevoglucosenone, dimethyl propylene urea and a mixtures thereof. It is preferred that said enhancer is methyl laurate. It has been surprisingly found that the use of such further enhancers may increase the release characteristic, e.g. by providing a permeation-enhancing effect, and, at the same time, that merely one of these enhancers is needed, which against helps to reduce the complexity of the TTS.

According to certain embodiments, the selexipag-containing layer further comprises an enhancer in the range from about 2.0 to about 15.0%, more preferably from about 3.0 to about 8.0% by, and in particular from 4.0 to about 6.0% by weight based on the total weight of the selexipag-containing layer. Alternatively or in addition, a mass ratio of the mass of selexipag to the mass of the enhancer in the selexipag-containing layer ranges from about 0.01 to about 3.0, preferably from about 0.1 to about 2.0; more preferably from about 0.1 to about 1.5, or from about 0.1 to about 0.9, or from about 0.5 to about 1.5.

The selexipag-containing layer may be a selexipag-containing matrix layer or a selexipag-containing reservoir layer. It is preferred that the selexipag-containing layer is a selexipag-containing matrix layer, which comprises selexipag homogeneously dispersed or dissolved in the polymer matrix. In another preferred embodiment, the selexipag-containing layer is a selexipag-containing biphasic matrix layer, which comprises an inner phase comprising the therapeutically effective amount of selexipag, and an outer phase comprising the at least one polar polymer and the at least one nonpolar polymer, wherein the inner phase forms dispersed deposits in the outer phase. The content of the inner phase in the biphasic matrix layer may be from about 5% to 40% by volume based on the volume of the biphasic matrix layer.

According to certain preferred embodiments, the selexipag-containing layer is a self-adhesive selexipag-containing matrix layer.

In a certain embodiment, the selexipag-containing layer is obtainable by coating and drying a selexipag-containing coating composition that comprises the selexipag, preferably by coating and drying a selexipag-containing coating composition, which comprises the at least one polar polymer and the at least one nonpolar polymer and the therapeutically effective amount of selexipag.

According to certain embodiments, the area weight of the selexipag-containing layer ranges from about 50 to about 250 g/m², preferably from about 70 to about 220 g/m², more preferably from about 80 to about 180 g/m², and particular from about 95 to about 160 g/m².

According to certain embodiments, a content of the at least one polar polymer in the selexipag-containing layer ranges from about 5 to about 80%, preferably from about 10 to about 50% or from about 5 to about 30%, more preferably from about 15 to about 40%, even more preferably from about 15 to about 30%, still more preferably of about 15 to about 27%, and in particular of about 15 to about 25%, by weight based on the total weight of the selexipag-containing layer. Preferably, the at least one polar polymer is selected from the group consisting of an acrylic polymer, a polyvinylpyrrolidone, and a combination thereof. Further details regarding the at least one polar polymer according to the invention are provided further below.

According to certain embodiments, a content of the at least one nonpolar polymer in the selexipag-containing layer ranges from about 50 to about 90%, preferably from about 60 to about 85%, and in particular from about 70 to about 80%, by weight based on the total weight of the selexipag-containing layer. According to certain alternative embodiments, a content of the at least one nonpolar polymer in the selexipag-containing layer ranges from about 5 to about 40%, preferably from about 10 to about 30%, and in particular from about 12 to about 25%, by weight based on the total weight of the selexipag-containing layer. Preferably, the at least one nonpolar polymer is a styrenic polymer, or a polyisobutylene. Further details regarding the at least one nonpolar polymer according to the invention are provided further below.

According to certain embodiments, a mass ratio of the mass of selexipag to the combined mass of the at least one polar polymer and the at least one nonpolar polymer in the selexipag-containing layer ranges from about 1×10⁻³ to about 0.1, preferably from about 5×10⁻³ to about 9×10⁻² or from about 9×10⁻³ to about 7×10⁻². In this connection it is to be understood that the dry mass of the respective polymers is considered, i.e. without a solvent.

According to certain embodiments, a mass ratio of the mass of the at least one polar polymer to the mass of the nonpolar polymer in the selexipag-containing layer ranges from about 0.01 to about 10.0, preferably from about 0.1 to 5.0; or from about 0.2 to about 2.0, preferably from about 0.2 to about 0.8. In this connection it is to be understood that the dry mass of the respective polymers is considered, i.e. without a solvent.

When using an additional skin contact layer, the ingredients of the selexipag-containing layer such as the selexipag and optional additional active agents, optional auxiliary polymers, optional anti-oxidants, and optional additional excipients or additives such as enhancers may over time migrate into the additional skin contact layer. This however depends on the ingredients and the material of the skin contact layer.

In one embodiment of the present invention, the selexipag-containing layer consists of a therapeutically active amount of selexipag, a polar polymer, and a nonpolar polymers. Alternatively, the selexipag-containing layer consists of a therapeutically active amount of selexipag, a polar polymer, a nonpolar polymer, and an enhancer as described herein.

In one embodiment of the present invention, in the selexipag-containing layer the at least one nonpolar polymer is a polyisobutylene polymer and/or a styrenic polymer; and the at least one polar polymer is an acrylic polymer and/or a polyvinylpyrrolidone. In a preferred embodiment, the at least one nonpolar polymer is a polyisobutylene polymer; and the at least one polar polymer is an acrylic polymer and/or a polyvinylpyrrolidone. In a further preferred embodiment, the at least one nonpolar polymer is a styrenic polymer; and the at least one polar polymer is a polyvinylpyrrolidone.

### Polar Polymer

The selexipag-containing layer according to the present invention comprises at least one polar polymer. The at least one polar polymer may be selected from the group consisting of an acrylic polymer, a polyvinylpyrrolidone, and mixtures thereof.

Preferably, a content of the at least one polar polymer in the selexipag-containing layer ranges from 5.0 to 30.0%, preferably from 15.0 to 27.0%, by weight based on the total weight of the selexipag-containing layer.

In a preferred embodiment, the at least one polar polymer is a polymer-based pressure-sensitive adhesive.

According to one embodiment of the invention, the at least one polar polymer is a polymer based on acrylates i.e. an acrylic polymer, preferably a pressure-sensitive adhesive based on acrylates. Pressure-sensitive adhesives based on acrylates may also be referred to as acrylate-based pressure-sensitive adhesives, or acrylate pressure-sensitive adhesives. Pressure-sensitive adhesives based on acrylates may have a solids content preferably between 30% and 60%. Such acrylate-based pressure-sensitive adhesives may or may not comprise functional groups such as hydroxy groups, carboxylic acid groups, neutralized carboxylic acid groups and mixtures thereof. Thus, the term “functional groups” in particular refers to hydroxy- and carboxylic acid groups, and deprotonated carboxylic acid groups.

Corresponding commercial products are available e.g. from Henkel under the tradename Duro-Tak®. Such acrylate-based pressure-sensitive adhesives are based on monomers selected from one or more of acrylic acid, butylacrylate, 2-ethylhexylacrylate, glycidylmethacrylate, 2-hydroxyethylacrylate, methylacrylate, methylmethacrylate, t-octylacrylamide and vinylacetate, and are provided in ethyl acetate, heptanes, n-heptane, hexane, methanol, ethanol, isopropanol, 2,4-pentanedione, toluene or xylene or mixtures thereof. Suitable acrylate-based pressure-sensitive adhesives are based on monomers selected from two or more of acrylic acid, butylacrylate, 2-ethylhexylacrylate, glycidylmethacrylate, 2-hydroxyethylacrylate, methylacrylate, methylmethacrylate, t-octylacrylamide and vinylacetate.

In one embodiment of the present invention, the at least one polar polymer may be an acrylic polymer and said acrylic polymer is a copolymer based on vinyl acetate, 2-ethylhexyl-acrylate, 2-hydroxyethyl-acrylate and optionally glycidyl-methacrylate.

Specific acrylate-based pressure-sensitive adhesives are available as:
Duro-Tak™ 387-2510 or Duro-Tak™ 87-2510 (a copolymer based on 2-ethylhexyl-acrylate, 2-hydroxyethyl-acrylate and methylacrylate, provided as a solution in ethyl acetate and hexane),
Duro-Tak™ 87-4287 (an acrylic copolymer comprising hydroxyl group, wherein the copolymer is based on vinyl acetate, 2-ethylhexyl-acrylate, and 2-hydroxyethyl-acrylate provided as a solution in ethyl acetate without cross-linking agent),
Duro-Tak™ 387-2287 or Duro-Tak™ 87-2287 (a copolymer based on vinyl acetate, 2-ethylhexyl-acrylate, 2-hydroxyethyl-acrylate and glycidyl-methacrylate provided as a solution in ethyl acetate without cross-linking agent),
Duro-Tak™ 387-2516 or Duro-Tak™ 87-2516 (a copolymer based on vinyl acetate, 2-ethylhexyl-acrylate, 2-hydroxyethyl-acrylate and glycidyl-methacrylate provided as a solution in ethyl acetate, ethanol, n-heptane and methanol with a titanium cross-linking agent),
Duro-Tak™ 387-2051 or Duro-Tak™ 87-2051 (a copolymer based on acrylic acid, butylacrylate, 2-ethylhexylacrylate and vinyl acetate, provided as a solution in ethyl acetate and heptane),
Duro-Tak™ 387-2353 or Duro-Tak™ 87-2353 (an acrylic copolymer comprising carboxylic acid groups, wherein the copolymer is based on acrylic acid, 2-ethylhexylacrylate, glycidylmethacrylate and methylacrylate, provided as a solution in ethyl acetate and hexane),
Duro-Tak™ 87-4098 (an acrylic copolymer comprising no functional groups, wherein the copolymer is based on 2-ethylhexyl-acrylate and vinyl acetate, provided as a solution in ethyl acetate),
Duro-Tak™ 87-9301 (an acrylic copolymer comprising no functional groups, obtained without using a crosslinking agent, provided in ethyl acetate),
Duro-Tak™ 87-2052 (a copolymer based on acrylic acid and vinyl acetate, comprising free carboxylic groups, obtained using a crosslinking agent, provided in ethyl acetate, heptane, ethanol, and isopropanol).

In a preferred embodiment, the at least one polar polymer is Duro-Tak™ 87-9301 (an acrylic copolymer comprising no functional groups, obtained without using a crosslinking agent, provided in ethyl acetate) or Duro-Tak™ 87-2052 (a copolymer based on acrylic acid and vinyl acetate, comprising free carboxylic groups, obtained using a crosslinking agent, provided in ethyl acetate, heptane, ethanol, and isopropanol), in particular Duro-Tak™ 87-2052.

In certain embodiment, the selexipag-containing layer according to the present invention comprises the acrylic polymer in an amount from about 10.0 to about 35.0%, preferably from about 20.0 to about 30.0%, by weight based on the total weight of the selexipag-containing layer.

In a certain embodiment, the at least one polar polymer may be a polyvinylpyrrolidone such as crospovidone. Preferably, the polyvinylpyrrolidone may be a soluble polyvinylpyrrolidone, but alternatively also an insoluble/cross-linked polyvinylpyrrolidone also known as crospovidones such as Kollidon® CL, Kollidon® CL-M and Kollidon® CL-SF, and polyvinylpyrrolidone-polyvinyl acetate copolymers, also known as crospovidones, such as Kollidon® VA64. Further suitable polyvinylpyrrolidones may be selected from the group consisting of Plasdone™ K-12 (having a weight average molecular weight of about 3,700 to 4,300 g/mol), Plasdone™ K-17 (having a weight average molecular weight of about 9,500 to 10,500 g/mol), Plasdone™ K-25 (having a weight average molecular weight of about 33,500 to 34,500 g/mol), Plasdone™ K-29/32 (having a weight average molecular weight of about 57,500 to 58,500 g/mol), Plasdone™ K-90 (having a weight average molecular weight of about 1,299,500 to 1,300,500 g/mol), Plasdone™ C-12 (having a weight average molecular weight of about 3,700 to 4,300 g/mol), Plasdone™ C-17 (having a weight average molecular weight of about 9,500 to 10,500 g/mol), and Plasdone™ C-30 (having a weight average molecular weight of about 57,500 to 58,500 g/mol). In this connection, the average molecular weight is preferably determined via SEC-MALS.

In certain embodiment, the selexipag-containing layer according to the present invention comprises polyvinylpyrrolidone, preferably crospovidone, in an amount from about 5.0 to about 30.0%, preferably from about 15.0 to about 27.0%, or from about 15.0 to about 25.0%, by weight based on the total weight of the selexipag-containing layer.

In certain embodiments, the selexipag-containing layer according to the present invention comprises at least two polar polymers. In this connection it is preferred that one polar polymer is an acrylic polymer and one polar polymer is a polyvinylpyrrolidone, preferably crospovidone.

### Nonpolar Polymer

The selexipag-containing layer according to the present invention comprises at least one nonpolar polymer. This also means that said selexipag-containing layer may also comprise a mixture of nonpolar polymers. Preferably, the at least one nonpolar polymer is selected from the group consisting of a silicone polymer, a polyisobutylene, a styrenic polymer, and mixtures thereof, and in particular is selected from the group consisting of a polyisobutylene, a styrenic polymer, and mixtures thereof. In a certain embodiment, the at least one nonpolar polymer is a pressure sensitive adhesive.

In one embodiment, said at least one nonpolar polymer is as silicone polymer. Silicone polymers are polymers based on polysiloxanes. These polymers based on polysiloxanes are preferably pressure sensitive adhesives based on polysiloxanes. Pressure-sensitive adhesives based on polysiloxanes may also be referred to as silicone-based pressure-sensitive adhesives, or silicone pressure sensitive adhesives.

According to a certain embodiment, the at least one nonpolar polymer is a polyisobutylene, or the at least one nonpolar polymer comprises a polyisobutylene mixture. In this connection, it is preferred that a content of the at least one nonpolar polymer in the selexipag-containing layer ranges from 50.0 to 90.0%, preferably from 60.0 to 85.0%, and in particular from 70.0 to 80.0%, by weight based on the total weight of the selexipag-containing layer.

In certain embodiments, the polyisobutylene is accessible by blending of 50 to 95%, preferably of 60 to 90%, and in particular of 75 to 90%, by weight, based on the total weight of the polyisobutylene with 5 to 50%, preferably 10 to 40%, and in particular 10 to 25%, by weight, based on the total weight of the polybutene. In this connection, it is preferred that the weight ratio of the mass of the polyisobutylene to the mass of the polybutene is of 20:1 to 1:1, and in particular of 8:1 to 3:1 Duro-Tak™ 87-6908 (having about 85% by weight of polyisobutylene and 15% by weight of polybutene, provided in n-heptane) provided by Henkel may be named as a suitable polyisobutylene. The solids content of polyisobutylenes in solvents is usually between 30 and 50%, preferably between 35 and 40%.

The at least nonpolar polymer may comprise a polyisobutylene mixture being a combination of a low molecular weight polyisobutylene and a high molecular weight polyisobutylene in a mass ratio of the mass of the low molecular weight polyisobutylene to the mass of the high molecular weight polyisobutylene ranging from 99:1 to 50:50, and wherein preferably the low molecular weight polyisobutylene has a viscosity average molecular weight of from 38,000 to 42,000 g/mol and/or a weight average molecular weight of from 34,000 to 40,000 g/mol, and wherein the high molecular weight polyisobutylene has a viscosity average molecular weight of from 1,100,000 to 1,120,000 g/mol and/or a weight average molecular weight of from 1,540,000 to 1,560,000 g/mol.

Suitable polyisobutylenes according to the invention are available under the tradename Oppanol®. Combinations of high-molecular weight polyisobutylenes (N100/N80) and low-molecular weight polyisobutylenes (B10, B11, B12, B13) may be used. Suitable ratios of low-molecular weight polyisobutylene to high-molecular weight polyisobutylene are in the range of from 100:1 to 1:100, preferably from 95:5 to 40:60, more preferably from 90:10 to 80:20. A preferred example for a polyisobutylene combination is B10/N100 in a ratio of 85/15. Oppanol® N100 has a viscosity average molecular weight Mᵥ of 1,110,000, and a weight average molecular weight M_{w} of 1,550,000, and an average molecular weight distribution M_{w}/Mₙ of 2.9. Oppanol® B10 has a viscosity average molecular weight Mᵥ of 40,000, and a weight average molecular weight M_{w} of 53,000, and an average molecular weight distribution M_{w}/Mₙ of 3.2. In certain embodiments, polybutene may be added to the polyisobutylenes. The solids content of polyisobutylenes in solvents is usually between 30 and 50%, preferably between 35 and 40%. The skilled person is aware that the solids content may be modified by adding a suitable amount of solvent.

According to another embodiment, the at least one nonpolar polymer is a styrenic polymer, preferably a styrenic thermoplastic elastomer. In this connection, it is preferred that a content of the at least one nonpolar polymer in the selexipag-containing layer ranges from 5.0 to 40.0%, preferably from 10.0 to 30.0%, and in particular from 12.0 to 25.0%, by weight based on the total weight of the selexipag-containing layer.

Suitable styrenic thermoplastic elastomers are styrene-isoprene block copolymers, preferably styrene-isoprene-styrene block copolymers. In this connection, it is preferred if the mass of the styrene to the mass of the isoprene is of 1:20 to 1:2, preferably of 1:15 to 1:3. JSR SIS5002, JSR SIS5229, JSR SIS5250, JSR SIS5403, and JSR SIS5505 (from JSR Life Science) shall be named as suitable styrene-isoprene-styrene block copolymer.

Preferably, the styrene-isoprene block copolymers comprises styrene units from 5 to 30%, preferably from 10 to 20%, by weight, based on the total amount of the styrene-isoprene block copolymers.

Preferably, the styrene-isoprene block copolymers has a melt flow rate (determined according to JIS K7210, 200° C., 49.0N) of 1 to 30 g/10 min, preferably of 5 to 20 g/10 min, and in particular of 6 to 15 g/10 min.

Further suitable styrenic thermoplastic elastomers are styrene-butadiene block copolymer. In this connection, it is preferred if the mass of the styrene to the mass of the butadiene is of 1:5 to 2:1, preferably of 1:3 to 1.1:1. JSR TR2000, JSR TR2001, JSR TR2250, JSR TR2500, JSR TR2630, JSR TR2787, JSR TR1084, and JSR TR1600 (from JSR Life Science) shall be named as suitable styrene-isoprene block copolymer.

Preferably, the styrene-isoprene block copolymer comprises styrene units from 30 to 60%, preferably from 35 to 55%, by weight, based on the total amount of the styrene-isoprene block copolymer.

Preferably, the styrene-isoprene block copolymer has a melt flow rate (determined according to JIS K7210, 200° C., 49.0N) of 1 to 25 g/10 min, preferably of 5 to 20 g/10 min, and in particular of 6 to 15 g/10 min.

In certain embodiments, the selexipag-containing layer according to the present invention comprises a tackifier. Preferably, the tackifier is a thermoplastic ester resin, and in particular a thermoplastic ester resin derived from glycerol and a highly stabilized rosin. Preferably, the tackifier has an acid number (mg KOH/g; determined according to ASTM D465) of 3 to 12, preferably of 4 to 10, and in particular of 5 to 9.

According to the present invention, the term “rosin” refers to a natural product obtained from the exudation of trees of the family Pinus. Rosin consists of about 90% by weight or more based on the total weight of the rosin, of mixed unsaturated acids. The rosin acids are mainly monobasic carboxylic acids, all containing the phenanthrene skeleton and all having 20 carbon atoms in the molecule, the main difference between the acids being the number and position of the double bonds. The acids can have conjugated double bonds like in abietic acid or a non-conjugated double bond as in pimaric acid. Natural rosin may be classified in several types due to its origin. There are, gum rosin, wood rosin and tall oil rosin (for further information see e.g. “Modern Surface Coatings” Paul Nylen et al, Interscience Publishers 1965, p 133-137). Because of its structure, rosin is generally unsuited for use in adhesive formulations. The double bonds of the rosin acids result in instability against oxidation and the isomer composition causes the rosin to crystallize. Therefore rosin must be subjected to chemical reactions altering the double bond position and changing the rosin acid isomer composition. Processes for stabilizing rosin are well known (stabilizing by hydrogenation, dehydrogenation or disproportionation).

In a certain embodiment, the selexipag-containing layer comprises a styrenic polymer and further comprises a tackifier, preferably wherein the weight ratio of the mass of the styrenic polymer to the mass of the tackifier is of 1:5 to 1:1, preferably of 1:4 to 1:2 and/or wherein a content of the tackifier in the selexipag-containing layer ranges from 30.0 to 80.0%, preferably from 40.0 to 70.0%, and in particular from 45.0 to 60.0%, by weight based on the total weight of the selexipag-containing layer.

### Release Characteristics

The TTS in accordance with the invention are designed for transdermally administering selexipag to the systemic circulation for a predefined extended period of time (such as about 48 h, or about 84 h, or about 168 h).

According to certain embodiments, the TTS according to the present invention provides a cumulative permeated amount of selexipag as measured in a Franz diffusion cell with dermatomed human skin of 10 to 120 μg/cm², preferably of 20 to 115 μg/cm², and in particular of 25 to 55 μm/cm² or of 70 to 115 μm/cm² over a time period of 48 hours.

According to certain embodiments, the TTS according to the present invention provides a cumulative permeated amount of selexipag as measured in a Franz diffusion cell with dermatomed human skin of 20 to 130 μg/cm², preferably of 35 to 100 μg/cm², and in particular of 45 to 80 μm/cm² over a time period of 84 hours.

In a certain embodiment, the TTS according to the present invention provides a cumulative permeated amount of selexipag as measured in a Franz diffusion cell with dermatomed human skin 30 to 250 μg/cm², preferably of 65 to 230 μg/cm², and in particular of 65 to 130 μg/cm² or of 170 to 230 μg/cm² over a time period of 168 hours.

In a certain embodiment, the TTS according to the present invention provides a cumulative permeated amount of selexipag as measured in a Franz diffusion cell with dermatomed human skin of 40 to 200 μg/cm², preferably of 60 to 150 μg/cm², and in particular of 65 to 120 μm/cm² over a time period of 168 hours.

According to certain embodiments, the TTS according to the present invention provides a cumulative permeated amount of selexipag as measured in a Franz diffusion cell with dermatomed human skin of 80 to 260 μg/cm², preferably of 150 to 240 μg/cm², and in particular of 170 to 230 μm/cm² over a time period of 168 hours.

In further certain embodiments, the TTS according to the present invention provides selexipag as measured in a Franz diffusion cell with dermatomed human skin in an amount of 0.2 to 10 mg, preferably of 0.3 to 5 mg, and in particular of 0.4 to 2.0 mg over a time period of 168 hours.

### Medical Use/Method of Treatment

In accordance with a second aspect of the present invention, the TTS according to the present invention is for use in a method of treating a human patient. Such method of treating a human patient may comprise administering the TTS on the skin of the patient. It is preferred that the TTS according to the present invention is for use in a method of treatment of pulmonary arterial hypertension (PAH) or chronic thromboembolic pulmonary hypertension (CTEPH). In particular, such treatment comprises a step of administering the TTS to the skin of a human patient.

According to one aspect, the invention relates to the use of a TTS according to the present invention for the manufacture of a medicament for of treating a human patient.

According to another aspect, the present invention relates to a method of treating a human patient.

### Method of Manufacture

The selexipag-containing layer can be manufactured according any suitable method.

In a certain embodiment, the method of manufacture of a selexipag-containing layer comprises the steps of:
1) combining at least the components selexipag, the at least one polar polymer, and the at least one nonpolar polymers, in a solvent to obtain a coating composition;
2) coating the coating composition onto the backing layer or release liner or any intermediate liner; and
3) drying the coated coating composition to form the selexipag-containing layer.

In this method of manufacture, preferably in step 1) at least one enhancer is added.

In the above described method preferably the solvent is selected from alcoholic solvents, in particular methanol, ethanol, isopropanol and mixtures thereof, and from non-alcoholic solvents, in particular ethyl acetate, hexane, n-heptane, petroleum ether, toluene, and mixtures thereof, and more preferably is selected from methanol, n-heptane and ethyl acetate.

In certain embodiments, the at least one polar polymer and the at least one nonpolar polymer are provided in a solvent, which preferably is selected from the group consisting of ethyl acetate, n-heptane, methanol or ethanol with a solids content of from 30 to 60% by weight.

In step 3), drying is performed preferably at a temperature of from 40 to 90° C., more preferably from 50 to 80° C.

### EXAMPLES

The present invention will now be more fully described with reference to the accompanying examples. It should be understood, however, that the following description is illustrative only and should not be taken in any way as a restriction of the invention.

### EXAMPLE 1

### Coating Composition

The formulations 1 to 3 of the selexipag-containing coating compositions are summarized below.

TABLE 1

| | Formulation 1 | | | Formulation 2 | | | Formulation 3 | | |
|---|---|---|---|---|---|---|---|---|---|
| Ingredient | Amt | Solids* | Solids | Amt | Solids* | Solids | Amt | Solids* | Solids |
| (Trade Name) | [g] | [g] | [%] | [g] | [g] | [%] | [g] | [g] | [%] |
| Selexipag | 0.15 | 0.15 | 2.0 | 0.15 | 0.15 | 2.0 | 0.15 | 0.15 | 2.0 |
| Tetrahydrofurfuryl | 0.38 | 0.38 | 5.0 | 0.38 | 0.38 | 5.1 | 0.38 | 0.38 | 5.1 |
| alcohol | | | | | | | | | |
| polyethylene | | | | | | | | | |
| glycol ether | | | | | | | | | |
| Povidone K90F | 7.50 | 1.50 | 20.0 | | | — | 1.88 | 0.38 | 5.0 |
| Polyacrylate | 14.26 | 5.48 | 73.0 | | | — | | | — |
| adhesive in ethyl | | | | | | | | | |
| acetate | | | | | | | | | |
| Solids content of | | | | | | | | | |
| 39% by weight | | | | | | | | | |
| (DURO-TAK ™ | | | | | | | | | |
| 87-4287 from | | | | | | | | | |
| Henkel) | | | | | | | | | |
| Polyisobutylene | | | — | 12.95 | 4.88 | 65.1 | 10.50 | 3.96 | 52.7 |
| adhesive in n- | | | | | | | | | |
| heptane | | | | | | | | | |
| Solids content of | | | | | | | | | |
| 38% by weight | | | | | | | | | |
| (DURO-TAK ™ | | | | | | | | | |
| 87-6908 from | | | | | | | | | |
| Henkel) | | | | | | | | | |
| Polyacrylate | | | — | 5.58 | 2.09 | 27.8 | 7.04 | 2.64 | 35.2 |
| adhesive in ethyl | | | | | | | | | |
| acetate | | | | | | | | | |
| Solids content of | | | | | | | | | |
| 36.5% by weight | | | | | | | | | |
| (DURO-TAK ™ | | | | | | | | | |
| 87-9301 from | | | | | | | | | |
| Henkel) | | | | | | | | | |
| Ethyl acetate | 0.79 | — | — | — | — | — | — | — | — |
| Total | 23.08 | 7.50 | 100.0 | 19.06 | 7.50 | 100.0 | 19.95 | 7.51 | 100.0 |
| Area Weight | 105.7 | | | 164.5 | | | 154.2 | | |
| [g/m²] | | | | | | | | | |
| Loading API | 209.1 | | | 324.9 | | | 305.2 | | |
| [μg/cm²] | | | | | | | | | |

*weight without solvent (dry weight)

### Preparation of the Coating Composition

Form. 1: Selexipag was put into a vessel and enhancer Tetrahydrofurfuryl alcohol polyethylene glycol ether was added. Successively solvent ethyl acetate and a pre-solution of Povidone K90F (in ethyl alcohol) were mixed into to the slurry. After adding DURO-TAK 87-4287 to the mass it was allowed to stir for minimum 1 hour.

Form. 2: Selexipag was put into a vessel and enhancer Tetrahydrofurfuryl alcohol polyethylene glycol as well as the DURO-TAK-adhesives were added. The mass was allowed to stir for minimum 1 hour.

Form. 3: Selexipag was put into a vessel and enhancer Tetrahydrofurfuryl alcohol polyethylene glycol ether and a pre-solution of Povidone K90F (in ethyl alcohol) were mixed into to the slurry. After adding the DURO-TAK adhesives to the mass it was allowed to stir for minimum 1 hour.

### Coating of the Coating Composition

The resulting selexipag-containing coating composition was coated on a siliconized PET foil. The coated material was dried for 15 minutes at room temperature and 15 minutes at 60° C. and afterwards laminated to a beige PET backing. The area weight was determined.

### Preparation of the TTS (all Examples)

The individual systems (TTS) were then punched out from the selexipag-containing self-adhesive layer structure.

In specific embodiments a TTS as described above can be provided with an adhesive overlay, i.e. a further self-adhesive layer structure of larger surface area, preferably with rounded corners, comprising a pressure-sensitive adhesive matrix layer which is free of active ingredient and a preferably skin-colored backing layer. The TTSs are then punched out and sealed into pouches of the primary packaging material.

### Example 2

### Coating Composition

The formulations 4 to 7 of the selexipag-containing coating compositions are summarized below.

TABLE 2

| | Formulation 4 | | | Formulation 5 | | |
|---|---|---|---|---|---|---|
| Ingredient | Amt | Solids* | Solids | Amt | Solids* | Solids |
| (Trade Name) | [g] | [g] | [%] | [g] | [g] | [%] |
| Selexipag | 0.10 | 0.10 | 1.0 | 0.10 | 0.10 | 1.0 |
| Tetrahydrofurfuryl | 0.50 | 0.50 | 5.1 | | | — |
| alcohol | | | | | | |
| polyethylene | | | | | | |
| glycol ether | | | | | | |
| Levulinic acid | | | — | 0.50 | 0.50 | 5.0 |
| Crospovidone | 2.00 | 2.00 | 20.0 | 2.00 | 2.00 | 20.0 |
| Polyisobutylene | 19.63 | 7.40 | 73.9 | 19.63 | 7.40 | 74.0 |
| adhesive in n- | | | | | | |
| heptane | | | | | | |
| Solids content of | | | | | | |
| 38% by weight | | | | | | |
| (DURO-TAK ™ | | | | | | |
| 87-6908 from | | | | | | |
| Henkel) | | | | | | |
| Ethyl acetate | 1.79 | | | 1.79 | | |
| Isopropanol | 0.60 | | | 0.60 | | |
| n-Heptane | 0.39 | | | 0.39 | | |
| Total | 25.00 | 10.00 | 100.0 | 25.00 | 10.00 | 100.0 |
| Area Weight [g/m²] | 141.1 | | | 149.7 | | |
| Loading API | 141.0 | | | 149.4 | | |
| [μg/cm²] | | | | | | |

*weight without solvent (dry weight)

TABLE 3

| | Formulation 6 | | | Formulation 7 | | |
|---|---|---|---|---|---|---|
| Ingredient | Amt | Solids* | Solids | Amt | Solids* | Solids |
| (Trade Name) | [g] | [g] | [%] | [g] | [g] | [%] |
| Selexipag | 0.100 | 0.10 | 1.0 | 0.10 | 0.10 | 1.0 |
| Transcutol | 0.5 | 0.50 | 5.1 | | | — |
| Methyl laurate | | | — | 0.50 | 0.50 | 5.1 |
| Crospovidone | 2.00 | 2.00 | 20.1 | 2.00 | 2.00 | 20.2 |
| Polyisobutylene | 19.63 | 7.40 | 73.8 | 19.63 | 7.40 | 73.7 |
| adhesive in n- | | | | | | |
| heptane | | | | | | |
| Solids content of | | | | | | |
| 38% by weight | | | | | | |
| (DURO-TAK ™ | | | | | | |
| 87-6908 from | | | | | | |
| Henkel) | | | | | | |
| Ethyl acetate | 1.79 | | | 1.80 | | |
| Isopropanol | 0.60 | | | 0.60 | | |
| n-Heptane | 0.39 | | | 0.40 | | |
| Total | 25.00 | 10.00 | 100.0 | 25.00 | 10.00 | 100.0 |
| Area Weight | 146.4 | | | 145.3 | | |
| [g/m²] | | | | | | |
| Loading API | 146.4 | | | 145.6 | | |
| [μg/cm²] | | | | | | |

*weight without solvent (dry weight)

### Preparation of the Coating Composition

Selexipag was put into a vessel and the rsp. enhancer was added. Successively the solvents were mixed into to the slurry. After adding DURO-TAK 87-6908 to the mass it was allowed to stir for 1 hour. Finally, Crospovidone was added to the mass while stirring. The mass was allowed to stir for another 2 hours.

### Coating of the Coating Composition

See Example 1.

### Preparation of the TTS (all Examples)

See Example 1.

### Example 3

### Coating Composition

The formulations 8 and 9 of the selexipag-containing coating compositions are summarized below.

TABLE 4

| | Formulation 8 | | | Formulation 9 | | |
|---|---|---|---|---|---|---|
| Ingredient | Amt | Solids* | Solids | Amt | Solids* | Solids |
| (Trade Name) | [g] | [g] | [%] | [g] | [g] | [%] |
| Selexipag | 0.10 | 0.10 | 1.0 | 0.10 | 0.10 | 1.0 |
| Methyl laurate | 0.50 | 0.50 | 5.1 | | | — |
| Crospovidone | 2.00 | 2.00 | 20.0 | 2.00 | 2.00 | 20.5 |
| Polyisobutylene | 20.23 | 7.40 | 73.9 | 21.59 | 7.90 | 78.5 |
| adhesive in n- | | | | | | |
| heptane | | | | | | |
| Solids content of | | | | | | |
| 38% by weight | | | | | | |
| (DURO-TAK ™ | | | | | | |
| 87-6908 from | | | | | | |
| Henkel) | | | | | | |
| Ethyl acetate | 1.41 | | | 0.85 | | |
| Isopropanol | 0.48 | | | 0.28 | | |
| n-Heptane | 0.30 | | | 0.19 | | |
| Total | 25.02 | 10.00 | 100.0 | 25.01 | 10.00 | 100.0 |
| Area Weight [g/m²] | 143.6 | | | 146.0 | | |
| Loading API | 145.0 | | | 151.0 | | |
| [μg/cm²] | | | | | | |

*weight without solvent (dry weight)

### Preparation of the Coating Composition

Form. 8: Selexipag was put into a vessel and the enhancer methyl laurate was added. Successively the solvents were mixed into to the slurry. After adding DURO-TAK 87-6908 to the mass it was allowed to stir for 1 hour. Finally, Crospovidone was added to the mass while stirring. The mass was allowed to stir for another 2 hours.

Form. 9: Selexipag was put into a vessel and the solvents were successively added. After adding DURO-TAK 87-6908 to the mass it was allowed to stir for minimum 1 hour. Finally, Crospovidone was added to the mass while stirring. The mass was allowed to stir for another 2 hours.

### Coating of the Coating Composition

See Example 1.

### Preparation of the TTS (all Examples)

See Example 1.

### Example 4

### Coating Composition

The formulations 10 to 12 of the selexipag-containing coating compositions are summarized below.

TABLE 5

| | Formulation 10 | | | Formulation 11 | | | Formulation 12 | | |
|---|---|---|---|---|---|---|---|---|---|
| Ingredient | Amt | Solids* | Solids | Amt | Solids* | Solids | Amt | Solids* | Solids |
| (Trade Name) | [g] | [g] | [%] | [g] | [g] | [%] | [g] | [g] | [%] |
| Selexipag | 0.15 | 0.15 | 1.5 | 0.15 | 0.15 | 1.5 | 0.15 | 0.15 | 1.5 |
| Methyl laurate | 0.50 | 0.50 | 5.1 | | | | 0.50 | 0.50 | 5.1 |
| Crospovidone | 2.00 | 2.00 | 20.0 | 2.00 | 2.00 | 20.1 | 2.70 | 2.70 | 27.0 |
| Polyisobutylene | 20.08 | 7.35 | 73.4 | 21.49 | 7.85 | 78.4 | 18.17 | 6.65 | 66.4 |
| adhesive in n- | | | | | | | | | |
| heptane | | | | | | | | | |
| Solids content of | | | | | | | | | |
| 38% by weight | | | | | | | | | |
| (DURO-TAK ™ | | | | | | | | | |
| 87-6908 from | | | | | | | | | |
| Henkel) | | | | | | | | | |
| Ethyl acetate | 1.46 | | | 0.90 | | | 2.24 | | |
| Isopropanol | 0.49 | | | 0.30 | | | 0.75 | | |
| n-Heptane | 0.32 | | | 0.20 | | | 0.49 | | |
| Total | 25.00 | 10.00 | 100.0 | 25.04 | 10.00 | 100.0 | 25.00 | 10.00 | 100.0 |
| Area Weight | 101.9 | | | 104.9 | | | 104.7 | | |
| [g/m²] | | | | | | | | | |
| Loading API | 154.3 | | | 161.3 | | | 159.5 | | |
| [μg/cm²] | | | | | | | | | |

*weight without solvent (dry weight)

### Preparation of the Coating Composition

Form. 10/12: Selexipag was put into a vessel and the enhancer methyl laurate was added. Successively the solvents were mixed into to the slurry. After adding DURO-TAK 87-6908 to the mass it was allowed to stir for 1 hour. Finally, Crospovidone was added to the mass while stirring. The mass was allowed to stir for another 2 hours.

Form. 11: Selexipag was put into a vessel and the solvents were successively added. After adding DURO-TAK 87-6908 to the mass it was allowed to stir for minimum 1 hour. Finally, Crospovidone was added to the mass while stirring. The mass was allowed to stir for another 2 hours.

### Coating of the Coating Composition

See Example 1.

### Preparation of the TTS (all Examples)

See Example 1.

### Example 5

### Coating Composition

The formulations 13 to 15 of the selexipag-containing coating compositions are summarized below.

TABLE 6

| | Formulation 13 | | | Formulation 14 | | | Formulation 15 | | |
|---|---|---|---|---|---|---|---|---|---|
| Ingredient | Amt | Solids* | Solids | Amt | Solids* | Solids | Amt | Solids* | Solids |
| (Trade Name) | [g] | [g] | [%] | [g] | [g] | [%] | [g] | [g] | [%] |
| Selexipag | 0.10 | 0.10 | 1.0 | 0.10 | 0.10 | 1.0 | 0.10 | 0.10 | 1.0 |
| Methyl laurate | 0.50 | 0.50 | 5.0 | | | — | 0.50 | 0.50 | 5.1 |
| Crospovidone | 2.00 | 2.00 | 20.1 | 2.00 | 2.00 | 20.1 | 2.70 | 2.70 | 27.0 |
| Styrene-isoprene- | 19.99 | 7.4 | 73.9 | 21.34 | 7.90 | 78.9 | 18.10 | 6.70 | 66.9 |
| styrene block | | | | | | | | | |
| copolymer having | | | | | | | | | |
| a styrene/rubber | | | | | | | | | |
| ratio of 15 to 85 | | | | | | | | | |
| (JSR SIS5229 | | | | | | | | | |
| from JSR Life | | | | | | | | | |
| Science) | | | | | | | | | |
| Ethyl acetate | 0.79 | | | 0.24 | | | 1.55 | | |
| Isopropanol | 0.26 | | | 0.08 | | | 0.52 | | |
| n-Heptane | 0.17 | | | 0.05 | | | 0.34 | | |
| Total | 23.81 | 10.00 | 100.0 | 23.81 | 10.00 | 100.0 | 23.81 | 10.00 | 100.0 |
| Area Weight | 144.1 | | | 152.6 | | | 159.7 | | |
| [g/m²] | | | | | | | | | |
| Loading API | 149.7 | | | 159.8 | | | 164.3 | | |
| [μg/cm²] | | | | | | | | | |

*weight without solvent (dry weight)

### Preparation of the Coating Composition

Form. 13/15: Selexipag was put into a vessel and the enhancer methyl laurate was added. Successively the solvents were mixed into to the slurry. After adding styrene-isoprene-styrene block copolymer to the mass it was allowed to stir for 1 hour. Finally, Crospovidone was added to the mass while stirring. The mass was allowed to stir for another 2 hours.

Form. 14: Selexipag was put into a vessel and the solvents were successively added. After adding styrene-isoprene-styrene block copolymer to the mass it was allowed to stir for minimum 1 hour. Finally, Crospovidone was added to the mass while stirring. The mass was allowed to stir for another 2 hours.

### Coating of the Coating Composition

See Example 1.

### Preparation of the TTS (all Examples)

See Example 1.

### Example 6

### Coating Composition

The formulations 16 to 19 of the selexipag-containing coating compositions are summarized below.

TABLE 7

| | Formulation 16 | | | Formulation 17 | | |
|---|---|---|---|---|---|---|
| Ingredient | Amt | Solids* | Solids | Amt | Solids* | Solids |
| (Trade Name) | [g] | [g] | [%] | [g] | [g] | [%] |
| Selexipag | 0.15 | 0.15 | 1.5 | 0.30 | 0.30 | 3.0 |
| Methyl laurate | | | — | 0.50 | 0.50 | 5.0 |
| Crospovidone | 2.70 | 2.70 | 27.0 | 2.70 | 2.70 | 27.0 |
| Polyisobutylene | 19.54 | 7.15 | 71.5 | 17.76 | 6.50 | 65.0 |
| adhesive in n- | | | | | | |
| heptane | | | | | | |
| Solids content of | | | | | | |
| 38% by weight | | | | | | |
| (DURO-TAK ™ | | | | | | |
| 87-6908 from | | | | | | |
| Henkel) | | | | | | |
| Ethyl acetate | 1.68 | | | 2.41 | | |
| Isopropanol | 0.56 | | | 0.80 | | |
| n-Heptane | 0.37 | | | 0.53 | | |
| Total | 25.00 | 10.00 | 100.0 | 25.00 | 10.00 | 100.0 |
| Area Weight [g/m²] | 106.1 | | | 105.3 | | |
| Loading API | 158.7 | | | 316.4 | | |
| [μg/cm²] | | | | | | |

*weight without solvent (dry weight)

TABLE 8

| | Formulation 18 | | | Formulation 19 | | |
|---|---|---|---|---|---|---|
| Ingredient | Amt | Solids* | Solids | Amt | Solids* | Solids |
| (Trade Name) | [g] | [g] | [%] | [g] | [g] | [%] |
| Selexipag | 0.50 | 0.50 | 5.0 | 0.50 | 0.50 | 5.0 |
| Methyl laurate | 0.50 | 0.50 | 5.1 | 0.50 | 0.50 | 5.0 |
| Crospovidone | 2.70 | 2.70 | 27.0 | 2.70 | 2.70 | 27.0 |
| Polyisobutylene | 17.21 | 6.30 | 62.9 | 17.21 | 6.30 | 63.0 |
| adhesive in n- | | | | | | |
| heptane | | | | | | |
| Solids content of | | | | | | |
| 38% by weight | | | | | | |
| (DURO-TAK ™ | | | | | | |
| 87-6908 from | | | | | | |
| Henkel) | | | | | | |
| Ethyl acetate | 2.63 | | | 2.63 | | |
| Isopropanol | 0.88 | | | 0.88 | | |
| n-Heptane | 0.58 | | | 0.58 | | |
| Total | 25.00 | 10.00 | 100.0 | 25.00 | 10.00 | 100.0 |
| Area Weight [g/m²] | 109.4 | | | 150.5 | | |
| Loading API | 546.5 | | | 753.1 | | |
| [μg/cm²] | | | | | | |

*weight without solvent (dry weight)

### Preparation of the Coating Composition

Form. 16: Selexipag was put into a vessel and the solvents were successively added. After adding DURO-TAK 87-6908 to the mass it was allowed to stir for minimum 1 hour. Finally, Crospovidone was added to the mass while stirring. The mass was allowed to stir for another 2 hours.

Form. 17-19: Selexipag was put into a vessel and the enhancer methyl laurate was added. Successively, the solvents were mixed into to the slurry. After adding DURO-TAK 87-6908 to the mass it was allowed to stir for 1 hour. Finally, Crospovidone was added to the mass while stirring. The mass was allowed to stir for another 2 hours.

### Coating of the Coating Composition

See Example 1.

### Preparation of the TTS (all Examples)

See Example 1.

### Example 7

### Coating Composition

The formulations 20 to 25 of the selexipag-containing coating compositions are summarized below.

TABLE 9

| | Formulation 20 | | | Formulation 21 | | | Formulation 22 | | |
|---|---|---|---|---|---|---|---|---|---|
| Ingredient | Amt | Solids* | Solids | Amt | Solids* | Solids | Amt | Solids* | Solids |
| (Trade Name) | [g] | [g] | [%] | [g] | [g] | [%] | [g] | [g] | [%] |
| Selexipag | 0.50 | 0.50 | 5.0 | 0.50 | 0.50 | 5.0 | 0.50 | 0.50 | 5.0 |
| Methyl laurate | | | — | 0.50 | 0.50 | 5.1 | 0.50 | 0.50 | 5.2 |
| Crospovidone | | | — | | | — | 1.00 | 1.00 | 10.1 |
| Polyisobutylene | 16.96 | 6.65 | 66.5 | 16.07 | 6.30 | 63.0 | 14.29 | 5.60 | 55.7 |
| adhesive in n- | | | | | | | | | |
| heptane | | | | | | | | | |
| Solids content of | | | | | | | | | |
| 38% by weight | | | | | | | | | |
| (DURO-TAK ™ | | | | | | | | | |
| 87-6908 from | | | | | | | | | |
| Henkel) | | | | | | | | | |
| Polyacrylate | 6.12 | 2.85 | 28.5 | 5.79 | 2.70 | 26.8 | 5.15 | 2.40 | 24.0 |
| adhesive in ethyl | | | | | | | | | |
| acetate and | | | | | | | | | |
| heptane | | | | | | | | | |
| Solids content of | | | | | | | | | |
| 47.5% by weight | | | | | | | | | |
| (DURO-TAK ™ | | | | | | | | | |
| 87-2052 from | | | | | | | | | |
| Henkel) | | | | | | | | | |
| n-Heptane | 1.42 | | | 2.14 | | | 3.56 | | |
| Total | 25.00 | 10.00 | 100.0 | 25.00 | 10.00 | 99.9 | 25.00 | 10.00 | 100.0 |
| Area Weight | 155.3 | | | 147.6 | | | 142.7 | | |
| [g/m²] | | | | | | | | | |
| Loading API | 778.5 | | | 744.1 | | | 718.1 | | |
| [μg/cm²] | | | | | | | | | |

*weight without solvent (dry weight)

TABLE 10

| | Formulation 23 | | | Formulation 24 | | | Formulation 25 | | |
|---|---|---|---|---|---|---|---|---|---|
| Ingredient | Amt | Solids* | Solids | Amt | Solids* | Solids | Amt | Solids* | Solids |
| (Trade Name) | [g] | [g] | [%] | [g] | [g] | [%] | [g] | [g] | [%] |
| Selexipag | 0.50 | 0.50 | 5.0 | 0.50 | 0.50 | 5.0 | 0.50 | 0.50 | 5.0 |
| Methyl laurate | | | — | 0.50 | 0.50 | 5.0 | 0.50 | 0.50 | 5.0 |
| Crospovidone | | | — | | | — | 1.00 | 1.00 | 10.1 |
| Polyisobutylene | 16.96 | 6.65 | 66.5 | 16.07 | 6.3 | 63.0 | 14.29 | 5.60 | 55.9 |
| adhesive in n- | | | | | | | | | |
| heptane | | | | | | | | | |
| Solids content of | | | | | | | | | |
| 38% by weight | | | | | | | | | |
| (DURO-TAK ™ | | | | | | | | | |
| 87-6908 from | | | | | | | | | |
| Henkel) | | | | | | | | | |
| Polyacrylate | 7.79 | 2.85 | 28.5 | 7.38 | 2.70 | 27.0 | 5.15 | 2.40 | 24.0 |
| adhesive in ethyl | | | | | | | | | |
| acetate | | | | | | | | | |
| Solids content of | | | | | | | | | |
| 36.5% by weight | | | | | | | | | |
| (DURO-TAK ™ | | | | | | | | | |
| 87-9301 from | | | | | | | | | |
| Henkel) | | | | | | | | | |
| n-Heptane | 0.07 | | | 0.55 | | | 3.56 | | |
| Total | 25.32 | 10.00 | 100.0 | 25.00 | 10.00 | 100.0 | 25.00 | 10.00 | 100.0 |
| Area Weight | 150.2 | | | 147.1 | | | 139.0 | | |
| [g/m²] | | | | | | | | | |
| Loading API | 753.0 | | | 739.8 | | | 699.4 | | |
| [μg/cm²] | | | | | | | | | |

*weight without solvent (dry weight)

### Preparation of the Coating Composition

Form. 20/23: Selexipag was put into a vessel and the solvent n-heptane was added. After adding the DURO-TAK adhesives to the mass it was allowed to stir for minimum 1 hour.

Form. 21/24: Selexipag was put into a vessel and enhancer methyl laurate as well as solvent n-heptane were added. After adding the DURO-TAK adhesives to the mass it was allowed to stir for minimum 1 hour.

Form. 22/25: Selexipag was put into a vessel and enhancer methyl laurate as well as solvent n-heptane were added. After adding the DURO-TAK adhesives to the mass it was allowed to stir for minimum 1 hour. Finally, Crospovidone was added to the mass while stirring. The mass was allowed to stir for another 2 hours.

### Coating of the Coating Composition

See Example 1.

### Preparation of the TTS (all Examples)

See Example 1.

### Example 8

### Measurement of Skin Permeation

The permeated amount of selexipag and the corresponding skin permeation rates of TTS prepared according to Examples 1 to 7 were determined by in vitro experiments in accordance with the OECD Guideline (adopted Apr. 13, 2004) carried out with a 7.0 ml Franz diffusion cell. Split thickness human skin from cosmetic surgeries (female breast or abdomen, date of birth 1964, 1984, 1992, 1993) was used (Example 1: [female breast, date of birth unknown]; Example 2: [female abdomen, date of birth 1984]; Example 3: [female abdomen, date of birth 1992]; Example 4: [female abdomen, date of birth 1992]; Example 5: [female abdomen, date of birth 1992]; Example 6: [female abdomen, date of birth 1964]; and Example 7: [female abdomen, date of birth 1993]). A dermatome was used to prepare skin to a thickness of 500 μm, with an intact epidermis for all TTS. Die cuts with an area of 1.16 cm² were punched from the TTS. The permeated amount of selexipag in the receptor medium of the Franz diffusion cell (an aqueous buffer comprising 0.9% aqueous sodium chloride, 3% vanillin, 0.5% methyl-β-cyclodextrin, 30% acetonitrile) at a temperature of 32±1° C. was measured and the corresponding cumulative permeated amount and the skin permeation rate were calculated.

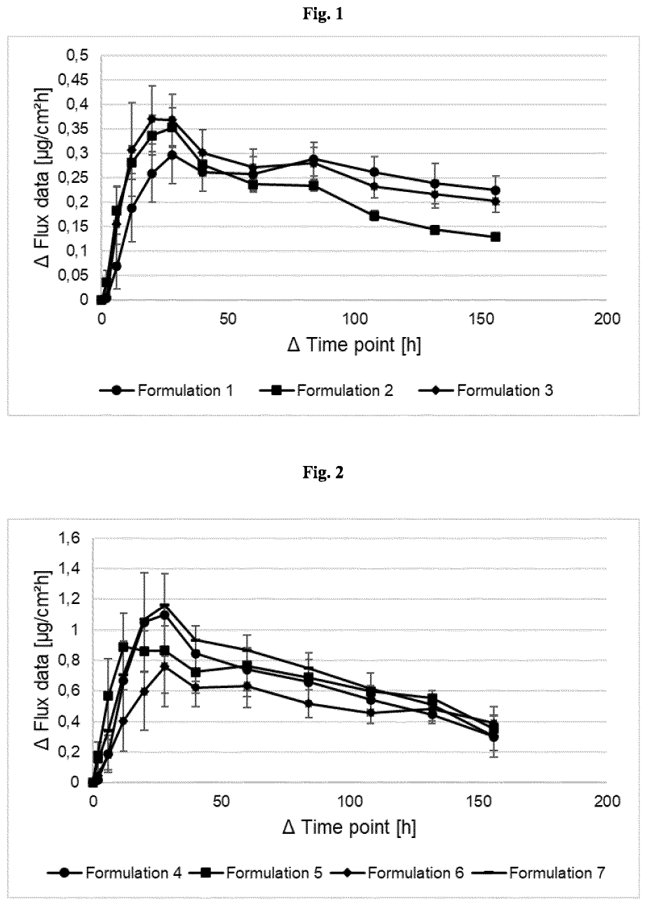

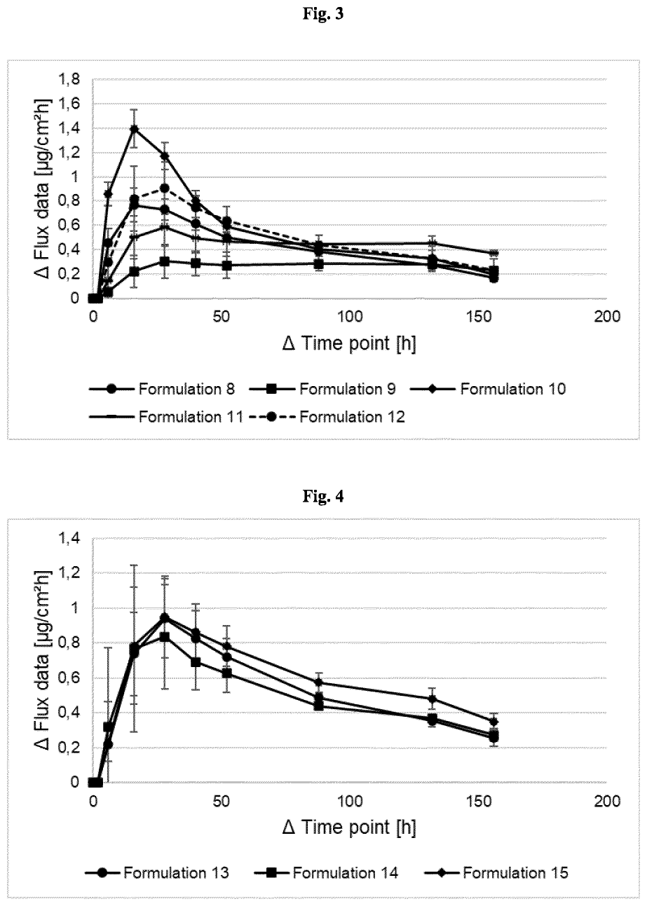

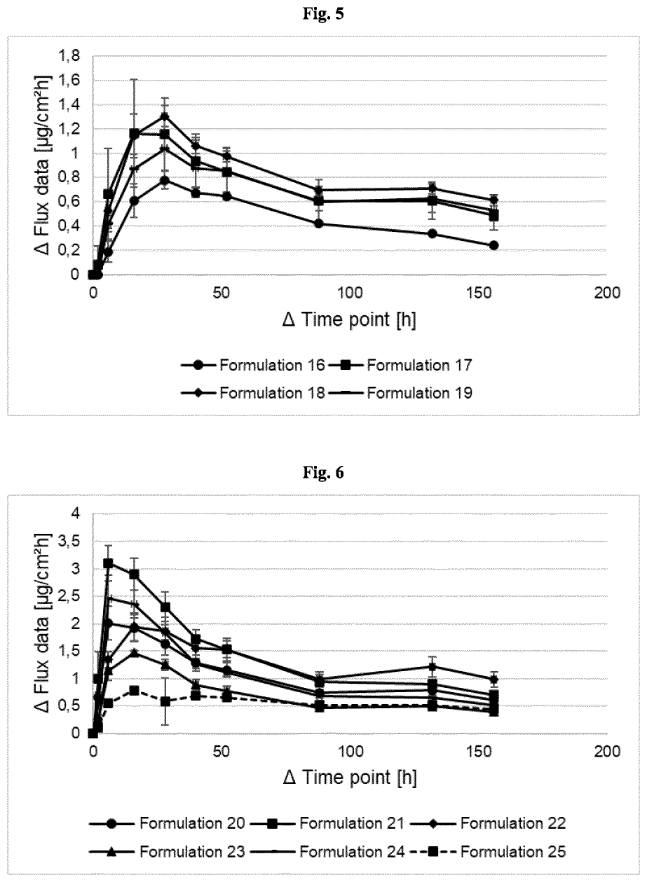

The results are shown in Table 11 below and in FIGS. 1 to 6.

TABLE 11

| | Permeation trial | Permeation trial | API Utilization |
|---|---|---|---|
| TTS | [μg/cm²]* | [μg/cm²]** | [μg/cm²]*** |
| Formulation 1 | 10.44 | 40.91 | 19.56 |
| Formulation 2 | 13.07 | 35.06 | 10.79 |
| Formulation 3 | 13.83 | 42.67 | 13.98 |
| Formulation 4 | 36.86 | 101.39 | 71.91 |
| Formulation 5 | 35.50 | 106.49 | 71.27 |
| Formulation 6 | 24.96 | 84.40 | 57.64 |
| Formulation 7 | 40.36 | 113.52 | 77.99 |
| Formulation 8 | 29.80 | 69.20 | 47.71 |
| Formulation 9 | 10.86 | 43.72 | 28.96 |
| Formulation 10 | 47.94 | 91.25 | 59.15 |
| Formulation 11 | 21.27 | 73.29 | 45.28 |
| Formulation 12 | 33.48 | 80.04 | 50.20 |
| Formulation 13 | 33.41 | 84.98 | 56.76 |
| Formulation 14 | 31.30 | 79.85 | 49.98 |
| Formulation 15 | 34.82 | 97.63 | 59.42 |
| Formulation 16 | 27.53 | 73.57 | 46.36 |
| Formulation 17 | 45.79 | 117.60 | 37.17 |
| Formulation 18 | 47.82 | 131.84 | 24.13 |
| Formulation 19 | 37.88 | 110.73 | 14.70 |
| Formulation 20 | 74.93 | 164.88 | 21.18 |
| Formulation 21 | 108.86 | 219.60 | 29.51 |
| Formulation 22 | 77.20 | 204.89 | 28.53 |
| Formulation 23 | 53.30 | 110.43 | 14.67 |
| Formulation 24 | 84.81 | 165.33 | 22.35 |
| Formulation 25 | 30.75 | 91.73 | 13.12 |

*Cumulative permeated amount after 48 hours.
**Cumulative permeated amount after 168 hours.
***API utilization rate after 168 hours.

### Example 9

### Formulation of Compositions

For determination of Selexipag and the Selexipag derived active metabolite MRE-269 permeated from fresh human skin tissue the following experiment was performed.

Modified Keshary-Chien diffusion cells were used for this experiment, in which fresh human skin was used as barrier between the donor and the acceptor part. The acceptor compartment contained the acceptor medium (0.9% NaCl+3% Vanillin+0.5% Methyl-β-cyclodextrin+30% Acetonitrile in aqua purificata), the donor chamber contained a liquid solution of Selexipag in oleic acid, dipropylene glycol, ethanol and aqua purificata. The liquid solution Composition 1 was applied as suspension, with undissolved Selexipag, whereas Composition 2 was centrifuged prior to application and only the clear centrifugate was applied. The composition of the selexipag-containing coating Compositions 1 and 2 of are summarized below.

TABLE 2

| | 1 | 2 |
|---|---|---|
| LTS REF | Composition 1 | Composition 2 |
| Selexipag | 4.99% | 2.51% |
| Oleic Acid | 9.00% | 7.97% |
| Dipropylene | 8.08% | 7.98% |
| glycol | | |
| Ethanol | 62.97% | 65.27% |
| Aqua purificata | 15.97% | 16.27% |
| Concentration | 3038.5 μg/cm² | 239.3 μg/cm² |
| Selexipag | | |

Fresh human skin from plastic surgery was used (upper arm, female, age 31). Directly after surgery the skin was prepared to remove fatty tissue, then placed in tissue wetted in perfusion solution and wrapped in sterile gauze. Shipping occurred on ice (on the same day), to keep the skin tissue cool, but not frozen to maintain physiological activity. Upon arrival the skin was dabbed dry and dermatomed to 500 μm without prior freezing. Lastly the skin tissue was punched out to a sample size of 3.843 cm².

A ring of adhesive foam (outer diameter of 27 mm and inner diameter of 18 mm) was applied on top of skin. This composite was placed to the acceptor compartment of the diffusion cell. The donor compartment was placed on top and fixed with a clamp to the acceptor chamber. The donor compartment was filled with approx. 94 mg of the liquid solutions, the acceptor compartment was filled with 10 mL of acceptor medium. The diffusion cells were incubated in a water bath (32° C.±0.5° C.) with continuous stirring (including a magnetic stirrer in the acceptor compartment) of 700 rpm to allow a homogeneous temperature and API concentration distribution. After 24-48-120-144-168 hrs the acceptor solution was fully removed, and the cells were refilled with fresh acceptor.

The samples of acceptor solution were filtrated with syringe filters and analyzed with HPLC. Quantification of Selexipag was performed with an external standard. The concentration of the active metabolite MRE-269 was recalculated with the response factor between Selexipag and the active metabolite MRE-269.

After 168 hrs of permeation the diffusion cells were dismantled, and the skin was rinsed with aqua purificata to remove residuals of the liquid solutions. Extraction of Selexipag and MRE-269 from the skin was performed by overnight shaking in methanol. The extracts were then diluted with acceptor medium, filtrated and analyzed with HPLC analogue to the permeation samples.

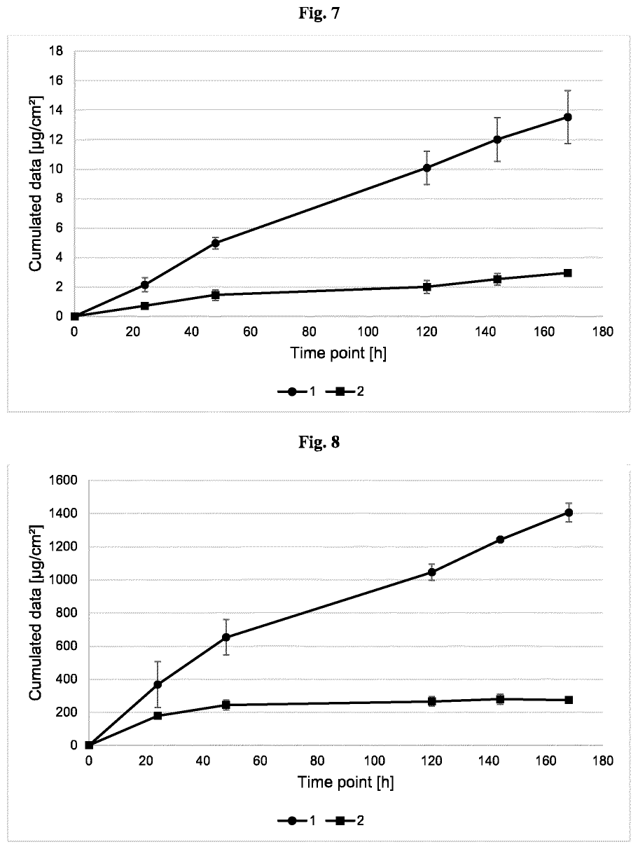

The active metabolite MRE-269 was detected at each sampling timepoint in all permeation samples as well as in the skin extraction samples. FIG. 7 shows the cumulative data of active metabolite MRE-269 in acceptor medium. A continuous release of active metabolite over 7 days has occurred, which proves the general presence of the active enzyme, responsible for metabolization of Selexipag in the skin, and its continuous activity over 7 days under in vitro conditions. Compared to the cumulative data of Selexipag in acceptor medium (FIG. 8) the release of MRE-269 starts slower, which fits to the metabolization that takes place prior.

The invention relates in particular to the following further items:

1. Transdermal therapeutic system for the transdermal administration of selexipag comprising a self-adhesive selexipag-containing layer structure, said self-adhesive selexipag-containing layer structure comprising:
A) a backing layer;
B) a selexipag-containing layer comprising:
   1. a therapeutically effective amount of selexipag;
   2. at least one polar polymer; and
   3. at least one nonpolar polymer.

2. Transdermal therapeutic system according to item 1, wherein a content of selexipag in the selexipag-containing layer ranges from about 0.1 to about 8.0%, preferably from about 0.5 to about 5.5%, by weight based on the total weight of the selexipag-containing layer 3. Transdermal therapeutic system according to item 1 or 2, wherein the mass ratio of the mass of selexipag to the combined mass of the at least one polar polymer and the at least one nonpolar polymer in the selexipag-containing layer ranges from about 1×10⁻³ to about 0.1, preferably from about 5×10⁻³ to about 9×10⁻² or from about 9×10⁻³ to about 7×10⁻².

4. Transdermal therapeutic system according to any one of items 1 to 3, wherein the selexipag-containing layer further comprises an enhancer.

5. Transdermal therapeutic system according to any one of items 1 to 4, wherein the selexipag-containing layer further comprises an enhancer selected from the group consisting of tetrahydrofurfuryl alcohol polyethylene glycol ether, levulinic acid, transcutol, lauryl lactate, methyl laurate, dihydrolevoglucosenone, dimethyl propylene urea and a mixtures thereof, and in particular methyl laurate.

6. Transdermal therapeutic system according to any one of items 1 to 5, wherein the selexipag-containing layer further comprises an enhancer in the range from about 2.0 to about 15.0%, more preferably from about 3.0 to about 8.0% by weight based on the total weight of the selexipag-containing layer.

7. Transdermal therapeutic system according to any one of items 4 to 6, wherein the mass ratio of the mass of selexipag to the mass of the enhancer in the selexipag-containing layer ranges from about 0.01 to about 3.0, preferably from about 0.1 to about 2.0; more preferably from about 0.1 to about 1.5, or from about 0.1 to about 0.9, or from about 0.5 to about 1.5.

8. Transdermal therapeutic system according to any one of items 1 to 7, wherein the at least one nonpolar polymer is a pressure sensitive adhesive and/or wherein the least one nonpolar polymer is selected from the group consisting of a silicone polymer, a polyisobutylene polymer, a styrenic polymer, and mixtures thereof, preferably selected from a polyisobutylene polymer, a styrenic polymer, and mixtures thereof.

9. Transdermal therapeutic system according to any one of items 1 to 8, wherein the at least one nonpolar polymer is a polyisobutylene mixture being a combination of polyisobutylene and polybutene, preferably wherein the weight ratio of the mass of the polyisobutylene to the mass of the polybutene is of about 20:1 to about 1:1, and in particular of about 8:1 to about 3:1.

10. Transdermal therapeutic system according to any one of items 1 to 9, wherein the at least one nonpolar polymer is a styrenic polymer, preferably a styrene-isoprene-styrene block copolymer, more preferably wherein the weight ratio of the mass of the styrene to the mass of the isoprene is of about 1:20 to about 1:2, preferably of about 1:15 to about 1:3.

11. Transdermal therapeutic system according to item 10, wherein the selexipag-containing layer further comprises a tackifier, preferably a thermoplastic ester resin, and in particular a thermoplastic ester resin derived from glycerol and a highly stabilized rosin.

12. Transdermal therapeutic system according to item 10 or 11, wherein the selexipag-containing layer further comprises a tackifier, wherein the weight ratio of the mass of the styrenic polymer to the mass of the tackifier is of about 1:5 to about 1:1, preferably of about 1:4 to about 1:2 and/or wherein a content of the tackifier in the selexipag-containing layer ranges from about 30.0 to about 80.0%, preferably from about 40.0 to about 70.0%, and in particular from about 45.0 to about 60.0%, by weight based on the total weight of the selexipag-containing layer.

13. Transdermal therapeutic system according to any one of items 10 to 12, wherein the tackifier has an acid number (mg KOH/g; determined according to ASTM D465) of about 3 to about 12, preferably of about 4 to about 10, and in particular of about 5 to about 9.

14. Transdermal therapeutic system according to any one of items 1 to 13, wherein the at least one polar polymer is selected form the group consisting of acrylic polymer, polyvinylpyrrolidone, and mixtures thereof.

15. Transdermal therapeutic system according to any one of items 1 to 14, wherein the at least one polar polymer is polyvinylpyrrolidone, preferably crospovidone.

16. Transdermal therapeutic system according to any one of items 1 to 15, wherein the at least one polar polymer is an acrylic polymer, preferably an a copolymer based on acrylic acid and vinyl acetate, comprising free carboxylic groups.

17. Transdermal therapeutic system according to any one of items 1 to 16, wherein a content of the at least one nonpolar polymer in the selexipag-containing layer ranges from about 50.0 to about 90.0%, preferably from about 60.0 to about 85.0%, and in particular from about 70.0 to about 80.0%, by weight based on the total weight of the selexipag-containing layer or wherein a content of the at least one nonpolar polymer in the selexipag-containing layer ranges from about 5.0 to about 40.0%, preferably from about 10.0 to about 30.0%, and in particular from about 12.0 to about 25.0%, by weight based on the total weight of the selexipag-containing layer.

18. Transdermal therapeutic system according to any one of items 1 to 17, wherein a content of the at least one polar polymer, preferably acrylic polymer, in the selexipag-containing layer ranges from about 10.0 to about 35.0%, preferably from about 20.0 to about 30.0%, by weight based on the total weight of the selexipag-containing layer.

19. Transdermal therapeutic system according to any one of items 1 to 17, wherein a content of the at least one polar polymer, preferably polyvinylpyrrolidone, in the selexipag-containing layer ranges from about 5.0 to about 30.0%, preferably from about 15.0 to about 27.0%, or from about 15.0 to about 25.0%, by weight based on the total weight of the selexipag-containing layer.

20. Transdermal therapeutic system according to any one of items 1 to 19, wherein the mass ratio of the mass of the polar polymer to the mass of the nonpolar polymer in the selexipag-containing layer ranges from about 0.01 to about 10.0, preferably from about 0.1 to 5.0; or from about 0.2 to about 2.0, preferably from about 0.2 to about 0.8.

21. Transdermal therapeutic system according to any one of items 1 to 20, wherein the at least one nonpolar polymer is a polyisobutylene polymer and/or a styrenic polymer; and wherein the at least one polar polymer is an acrylic polymer and/or a polyvinylpyrrolidone.

22. Transdermal therapeutic system according to any one of items 1 to 21, wherein the at least one nonpolar polymer is a polyisobutylene polymer; and wherein the at least one polar polymer is an acrylic polymer and/or a polyvinylpyrrolidone.

23. Transdermal therapeutic system according to any one of items 1 to 21, wherein the at least one nonpolar polymer is a styrenic polymer; and wherein the at least one polar polymer is a polyvinylpyrrolidone.

24. Transdermal therapeutic system according to any one of items 1 to 23, wherein the selexipag-containing layer is a selexipag-containing matrix layer; and/or wherein the area weight of the selexipag-containing layer ranges from about 70 to about 220 g/m², preferably from about 95 to about 160 g/m².

25. Transdermal therapeutic system according to any one of items 1 to 24, providing a cumulative permeated amount of selexipag as measured in a Franz diffusion cell with dermatomed human skin of 10 μg/cm² to 120 μg/cm², preferably of 20 μg/cm² to 115 μg/cm², and in particular of 25 to 55 μg/cm² or of 70 to 115 μg/cm² over a time period of 48 hours and/or providing a cumulative permeated amount of selexipag as measured in a Franz diffusion cell with dermatomed human skin of 30 μg/cm² to 250 μg/cm², preferably of 65 μg/cm² to 230 μg/cm², and in particular of 65 to 130 μg/cm² or of 170 to 230 μg/cm² over a time period of 168 hours.

26. Transdermal therapeutic system according to any one of items 1 to 25, wherein said selexipag-containing layer structure comprises:
A) a backing layer;
B) a selexipag-containing layer comprising:
   1. a therapeutically effective amount of selexipag;
   2. at least one polar polymer; and
   3. at least one nonpolar polymer.

27. Transdermal therapeutic system according to any one of items 1 to 25, wherein said selexipag-containing layer structure comprises:
A) a backing layer;
B) a selexipag-containing layer comprising:
   1. a therapeutically effective amount of selexipag;
   2. at least one polar polymer being a polyvinylpyrrolidone; and
   3. at least one nonpolar polymer, wherein the at least one nonpolar polymer comprises a polyisobutylene.

28. Transdermal therapeutic system according to any one of items 1 to 25, wherein said selexipag-containing layer structure comprises:
A) a backing layer;
B) a selexipag-containing layer comprising:
   1. a therapeutically effective amount of selexipag;
   2. at least one polar polymer;
   3. at least one nonpolar polymer; and
   4. at least one enhancer.

29. Transdermal therapeutic system according to any one of items 1 to 25, wherein said selexipag-containing layer structure comprises:
A) a backing layer;
B) a selexipag-containing layer comprising:
   1. a therapeutically effective amount of selexipag;
   2. at least one polar polymer;
   3. at least one nonpolar polymer; and
   4. at least one enhancer being methyl laurate.

30. Transdermal therapeutic system according to any one of items 1 to 25, wherein said selexipag-containing layer structure comprises:
A) a backing layer;
B) a selexipag-containing layer comprising:
   1. a therapeutically effective amount of selexipag;
   2. at least one polar polymer being an acrylic polymer; and
   3. at least one nonpolar polymer wherein the at least one nonpolar polymer comprises a polyisobutylene.

31. Transdermal therapeutic system according to any one of items 1 to 25, wherein said selexipag-containing layer structure comprises:
A) a backing layer;
B) a selexipag-containing layer comprising:
   1. a therapeutically effective amount of selexipag;
   2. at least one polar polymer being an acrylic polymer;
   3. at least one nonpolar polymer wherein the at least one nonpolar polymer comprises a polyisobutylene;
   4. at least one enhancer being methyl laurate.

32. Transdermal therapeutic system according to any one of items 1 to 31 for use in a method of treating a human patient.

33. Transdermal therapeutic system according to any one of items 1 to 31 for use in for use in a method of treatment of pulmonary arterial hypertension (PAH) or chronic thromboembolic pulmonary hypertension (CTEPH) in a human patient.

1. Transdermal therapeutic system for the transdermal administration of selexipag comprising a self-adhesive selexipag-containing layer structure, said self-adhesive selexipag-containing layer structure comprising:
A) a backing layer;
B) a selexipag-containing layer comprising:
1. selexipag;
2. at least one polar polymer; and
3. at least one nonpolar polymer.

2. Transdermal therapeutic system according to claim 1, wherein a content of selexipag in the selexipag-containing layer ranges from 0.1 to 8.0%, preferably from 0.5 to 5.5%, by weight based on the total weight of the selexipag-containing layer.

3. Transdermal therapeutic system according to claim 1 or 2, wherein the selexipag-containing layer further comprises an enhancer, preferably selected from the group consisting of tetrahydrofurfuryl alcohol polyethylene glycol ether, levulinic acid, transcutol, lauryl lactate, methyl laurate, dihydrolevoglucosenone, dimethyl propylene urea and a mixtures thereof, and in particular methyl laurate and/or wherein the selexipag-containing layer further comprises an enhancer in the range from 2.0 to 15.0%, more preferably from 3.0 to 8.0% by weight based on the total weight of the selexipag-containing layer.

4. Transdermal therapeutic system according to any one of claims 1 to 3, wherein the at least one nonpolar polymer is a pressure sensitive adhesive and/or wherein the least one nonpolar polymer is selected from the group consisting of a silicone polymer, a polyisobutylene polymer, a styrenic polymer, and mixtures thereof, preferably selected from a polyisobutylene polymer, a styrenic polymer, and mixtures thereof.

5. Transdermal therapeutic system according to any one of claims 1 to 4, wherein the at least one nonpolar polymer is a polyisobutylene mixture being a combination of polyisobutylene and polybutene, preferably wherein the weight ratio of the mass of the polyisobutylene to the mass of the polybutene is of 20:1 to 1:1, and in particular of 8:1 to 3:1.

6. Transdermal therapeutic system according to any one of claims 1 to 5, wherein the at least one nonpolar polymer is a styrenic polymer, preferably a styrene-isoprene-styrene block copolymer, more preferably wherein the weight ratio of the mass of the styrene to the mass of the isoprene is of 1:20 to 1:2, preferably of 1:15 to 1:3.

7. Transdermal therapeutic system according to claim 6, wherein the selexipag-containing layer further comprises a tackifier, preferably a thermoplastic ester resin, and in particular a thermoplastic ester resin derived from glycerol and a highly stabilized rosin.

8. Transdermal therapeutic system according to claim 6 or 7, wherein the selexipag-containing layer further comprises a tackifier,
wherein the weight ratio of the mass of the styrenic polymer to the mass of the tackifier is of 1:5 to 1:1, preferably of 1:4 to 1:2 and/or wherein a content of the tackifier in the selexipag-containing layer ranges from 30.0 to 80.0%, preferably from 40.0 to 70.0%, and in particular from 45.0 to 60.0%, by weight based on the total weight of the selexipag-containing layer.

9. Transdermal therapeutic system according to any one of claims 1 to 8, wherein the at least one polar polymer is selected form the group consisting of acrylic polymer, polyvinylpyrrolidone, and mixtures thereof.

10. Transdermal therapeutic system according to any one of claims 1 to 9, wherein the at least one polar polymer is polyvinylpyrrolidone, preferably crospovidone.

11. Transdermal therapeutic system according to any one of claims 1 to 10, wherein a content of the at least one nonpolar polymer in the selexipag-containing layer ranges from 50.0 to 90.0%, preferably from 60.0 to 85.0%, and in particular from 70.0 to 80.0%, by weight based on the total weight of the selexipag-containing layer or
wherein a content of the at least one nonpolar polymer in the selexipag-containing layer ranges from 5.0 to 40.0%, preferably from 10.0 to 30.0%, and in particular from 12.0 to 25.0%, by weight based on the total weight of the selexipag-containing layer.

12. Transdermal therapeutic system according to any one of claims 1 to 11, wherein a content of the at least one polar polymer, preferably polyvinylpyrrolidone, in the selexipag-containing layer ranges from 5.0 to 30.0%, preferably from 15.0 to 27.0%, by weight based on the total weight of the selexipag-containing layer.

13. Transdermal therapeutic system according to any one of claims 1 to 12, wherein the selexipag-containing layer is a selexipag-containing matrix layer; and/or wherein the area weight of the selexipag-containing layer ranges from 70 to 220 g/m², preferably from 95 to 160 g/m².

14. Transdermal therapeutic system according to any one of claims 1 to 13,
providing a cumulative permeated amount of selexipag as measured in a Franz diffusion cell with dermatomed human skin of 10 μg/cm² to 120 μg/cm², preferably of 20 μg/cm² to 115 μg/cm² over a time period of 48 hours and/or
providing a cumulative permeated amount of selexipag as measured in a Franz diffusion cell with dermatomed human skin of 30 μg/cm² to 250 μg/cm², preferably of 65 μg/cm² to 230 μg/cm² over a time period of 168 hours.

15. Transdermal therapeutic system according to any one of claims 1 to 14 for use in a method of treating a human patient.
